SEQUENCE LISTING (7 sequences)

SEQ ID NO: 1 - protein, 1368 aa, Artificial
  Synthetic pyogenes dCas9 Protein Sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - protein, 200 aa, Artificial
  Synthetic AID*delta
MDSLLMNRREFLYQFKNVRWAKGRRETYLCYVVKRRDSATSFSLDFGYLRNKNGCHVELLFLRYISDWDLDPGRCYRVTWFISWSPCYDCARHVADFLRGNPNLSLRIFTARLYFCEDRKAEPEGLRRLHRAGVQIAIMTFKDYFYCWNTFVENHGRTFKAWEGLHENSVRLSRQLRRILLPLYEVDDLRDAFRTCTGSG

SEQ ID NO: 3 - protein, 117 aa, Artificial
  Synthetic MS2 coat protein variant
MASNFTQFVLVDNGGYGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 4 - DNA, 25 bp, Artificial
  Synthetic MS2 Phage Operator Stem Loop
gcgcacatgaggatcacccatgtgc

SEQ ID NO: 5 - protein, 64 aa, Homo sapiens
IYPYDSDTQYSPSFQGQVTISADKSTTTAYLHWSSLKASDTAMYYCVRLRGGFVRVVFAPYFDS

SEQ ID NO: 6 - protein, 64 aa, Homo sapiens
  MISC_FEATURE (33)..(33): X is L or W
  MISC_FEATURE (43)..(43): X is I or M
  MISC_FEATURE (52)..(52): X is D, G or E
  MISC_FEATURE (55)..(55): X is P or R
IYPYDSDTQYSPSFQGQVTISADKSTTTAYLHXSSLKASDTAXYYCVRLRGXFVXVVFAPYFDS

SEQ ID NO: 7 - protein, 64 aa, Homo sapiens
IYPYHSDTQYTPSFQGQVTISADKSTTTAYLHLSSLKASDTAIYYCVRLRGDFVPVVFAPYFDS

(19) United States
(12) Patent Application Publication
(10) Pub. No.: US 2022/0106584 A1
(43) Pub. Date: Apr. 7, 2022

(54) METHODS AND KITS FOR GENERATING AND SELECTING A VARIANT OF A BINDING PROTEIN WITH INCREASED BINDING AFFINITY AND/OR SPECIFICITY

(71) Applicants: INSERM (Institut National de la Santé et de la Recherche Médicale), Paris (FR); Université de Nantes, Nantes (FR); Centre National de la Recherche Scientifique (CNRS), Paris (FR); Centre Hospitalier Universitaire de Nantes, Nantes (FR)

(72) Inventors: Xavier SAULQUIN, Nantes Cedex 1 (FR); Laetitia GAUTREAU-ROLLAND, Nantes Cedex 1 (FR); Richard BREATHNACH, Nantes Cedex 1 (FR); Mélinda MOYON, Nantes (FR); Marie-Claire DEVILDER, Nantes Cedex 1 (FR)

(21) Appl. No.: 17/421,175

(22) PCT Filed: Jan. 13, 2020
(86) PCT No.: PCT/EP2020/050646

§ 371 Date: Jul. 7, 2021

(30) Foreign Application Priority Data
Jan. 14, 2019 (EP) 19305040.8

(51) Int. Cl. C12N 15/10; C12N 15/113; C12N 9/22

(52) U.S. Cl. CPC C12N 15/1034; C12N 15/1138; C12N 2310/20; C12N 2310/3519; C12N 9/22

(57) ABSTRACT

Somatic hypermutation promotes affinity maturation of antibodies by targeting the cytidine deaminase AID to antibody genes, followed by antigen-based selection of matured antibodies. Given the importance of antibodies in medicine and research, developing approaches to reproduce this natural phenomenon in cell culture is of some interest. The inventors use here the CRISPR-Cas 9 based CRISPR-X approach to target AID to antibody genes carried by expression vectors in HEK 293 cells. This directed mutagenesis approach, combined with a highly sensitive antigen-associated magnetic enrichment process, allowed rapid progressive evolution of a human antibody against the Human Leucocyte Antigen A*0201 allele. Starting from a low affinity monoclonal antibody expressed on Ag-specific naïve blood circulating B cells, they obtained in approximately 6 weeks antibodies with a two log increase in affinity and which retained their specificity. The strategy for in vitro affinity maturation of antibodies is applicable to virtually any antigen. It not only allows to tap into the vast naive B cell repertoire but could also be useful when dealing with antigens that only elicit low affinity antibodies after immunization. Accordingly as defined by the claims, the present invention relates to methods and kits for generating and selecting a variant of antibody binding protein with increased binding affinity and/or specificity.

### FIELD OF THE INVENTION

The present invention relates to methods and kits for generating and selecting a variant of antibody binding protein with increased binding affinity and/or specificity.

### BACKGROUND OF THE INVENTION

The human B cell repertoire constitutes a source of antibodies capable of recognizing virtually any antigen (Ag). This is the result of a complex B lymphocyte maturation process. Newly produced B cells express B cell receptors (BCRs) generated by random somatic recombination of V (Variable), D (Diversity) and J (Junction) gene segments and which generally have a low affinity for their cognate Ag[1]. After exposure to an Ag, naïve B cells with Ag-specific BCRs undergo somatic hypermutation (SHM) catalyzed by the enzyme Activation induced cytidine deaminase (AID)[2-4]. This enzyme is targeted to the Ig-loci in B cells and deaminates cytosines, thus provoking point mutations, insertions and deletions in the variable domains of both the heavy and light chains. This process ultimately leads to antibody diversification and is followed by the selection of a matured B cell repertoire with higher affinity and specificity for the Ag. This allows the overall diversity of the BCR/antibody molecules to reach theoretically about 10¹³ different receptors in humans[5]. The repertoire thus constitutes an almost unlimited resource of antibodies.

For several decades, monoclonal antibodies (mAb) have been crucial tools in the treatment of diseases such as autoimmune diseases and cancer, or for the control of graft rejection. It is important to generate fully human mAbs because they have a lower risk of immune response induction in humans than the mouse, chimeric or humanized mAbs generally used hitherto. Various methods have been developed for isolating antibodies directly from a natural repertoire of human B lymphocytes. In general, they derive from two main approaches. The first of these is the high-throughput screening of mAb produced by B cell cultures or plasma cells[6, 7]. This is a very effective method for obtaining mAb against Ag to which an individual is exposed naturally or by vaccination. However, many Ag of therapeutic interest are not encountered sufficiently frequently naturally, or exploitable in vaccine strategies in humans, to profit from this type of methodology. The second technique consists in isolating single Ag-specific B cells using fluorescent-tagged Ag, followed by cloning of their immunoglobulin genes and expression of recombinant antibodies in a cell line. This technique allows interrogation of both the immune/matured B cell repertoire and the naïve/germline repertoire of an individual with respect to any Ag available in purified form[8-10]. There is a limitation to the interrogation of a naive B cell repertoire however: the generally limited affinity of the corresponding recombinant antibodies, requiring identification of mutations that enhance affinity while maintaining specificity.

Antibody optimization currently relies heavily on the use of libraries generated by mutagenesis of antibody chains using error-prone PCR or degenerate primers. Libraries are screened using techniques such as ribosome, phage, yeast or mammalian display[11]. Co-expression of AID and antibody or non-antibody genes in various mammalian cell lines has also been used to initiate a mutagenic process mimicking SHM[12-20]. This approach circumvents the need to construct mutant libraries, but does not allow targeting of the AID enzyme to sequences encoding the antibody. In B cells, AID is targeted to the immunoglobulin locus by complex mechanisms not yet fully elucidated[21].

Various CRISPR Cas9-based approaches using guide RNAs to target base editors such as APOBEC or AID fused to dead Cas9 (dCas9) to specific DNA sequences have been described recently[22, 23]. These approaches generally lead to mutations limited to a small part of the sequences corresponding to the guide RNA binding site. A variant approach (CRISPR-X) uses a complex containing dCas9 and a guide RNA containing bacteriophage MS2 coat protein binding sites to recruit a coat-AID fusion to DNA[24]. This leads to more extensive mutagenesis covering a window of approximately 100 bp around the guide RNA binding site.

### SUMMARY OF THE INVENTION

As defined by the claims, the present invention relates to methods and kits for generating and selecting a variant of antibody binding protein with increased binding affinity and/or specificity.

### DETAILED DESCRIPTION OF THE INVENTION

Somatic hypermutation promotes affinity maturation of antibodies by targeting the cytidine deaminase AID to antibody genes, followed by antigen-based selection of matured antibodies. Given the importance of antibodies in medicine and research, developing approaches to reproduce this natural phenomenon in cell culture is of some interest.

The inventors use here the CRISPR-Cas 9 based CRISPR-X approach to target AID to antibody genes carried by expression vectors in HEK 293 cells. This directed mutagenesis approach, combined with a highly sensitive antigen-associated magnetic enrichment process, allowed rapid progressive evolution of a human antibody against the Human Leucocyte Antigen A*0201 allele. Starting from a low affinity monoclonal antibody expressed on Ag-specific naïve blood circulating B cells, they obtained in approximately 6 weeks antibodies with a two log increase in affinity and which retained their specificity.

The strategy for in vitro affinity maturation of antibodies is applicable to virtually any antigen. It not only allows to tap into the vast naive B cell repertoire but could also be useful when dealing with antigens that only elicit low affinity antibodies after immunization.

### Main Definitions

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, pegylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, a “nucleic acid molecule” or “polynucleotide” refers to a DNA molecule (for example, but not limited to, a cDNA or genomic DNA). The nucleic acid molecule can be single-stranded or double-stranded.

The term “isolated” when referring to nucleic acid molecules or polypeptides means that the nucleic acid molecule or the polypeptide is substantially free from at least one other component with which it is associated or found together in nature.

As used herein, the term “complementarity” refers to the ability of a nucleic acid to form hydrogen bond(s) with another nucleic acid sequence by either traditional Watson-Crick base-pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y.

“Hybridization” or “hybridizing” refers to a process where completely or partially complementary nucleic acid strands come together under specified hybridization conditions to form a double-stranded structure or region in which the two constituent strands are joined by hydrogen bonds. Although hydrogen bonds typically form between adenine and thymine or uracil (A and T or U) or cytosine and guanine (C and G), other base pairs may form (e.g., Adams et al., The Biochemistry of the Nucleic Acids, 11th ed., 1992).

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, the term “binding protein” refers to a protein that can bind to a target molecule. The binding protein comprises a “binding domain” As used herein, the term “binding domain”, i.e. an amino acid sequence region that preferentially binds to the target molecule under physiological conditions. Binding proteins include not only antibodies but as well as any other proteins potentially capable of binding a given target molecule that typically include but are not limited to protein ligands and receptors.

The term “target molecule” refers to a molecule, usually a polypeptide, which is capable of being bound by a binding protein and has one or more binding sites for the binding protein. As used herein, the term “specific target molecule” refers to the target molecule that is bound by the binding protein of interest. Inversely, the term “non-specific target molecule” refers to a target molecule that is not bound by the binding protein of interest but that is bound by another binding protein.

As used herein, the term “specificity” refers to the ability of a binding protein (e.g. an antibody) to detectably bind target molecule (e.g. an epitope presented on an antigen) while having relatively little detectable reactivity with other target molecules. Specificity can be relatively determined by binding or competitive binding assays, using, e.g., Biacore instruments, as described elsewhere herein. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules.

The term “affinity”, as used herein, means the strength of the binding of a binding protein (e.g. an antibody) to a target molecule (e.g. an epitope). The affinity of a binding protein is given by the dissociation constant Kd. For an antibody said Kd is defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of a binding protein can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of binding protein is the use of Biacore instruments.

The term “binding” as used herein refers to a direct association between two molecules, due to, for example, covalent, electrostatic, hydrophobic, and ionic and/or hydrogen-bond interactions, including interactions such as salt bridges and water bridges. In particular, as used herein, the term “binding” in the context of the binding of a binding protein (e.g. an antibody) to a predetermined target molecule (e.g. an antigen or epitope) typically is a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less.

As used herein, the term “avidity” is meant to refer to a measure of the overall stability of the complex between binding proteins and their target molecules. The overall stability of the interaction can be governed by three major factors as follows: (a) the intrinsic affinity of binding protein for the target molecule; (b) the valency of the binding protein and the target molecule; and (c) the geometric arrangement of the interacting components. As such, the avidity of the complex can be modulated by varying the foregoing parameters, as well as others.

As used herein the term “antibody” or “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments (e.g., Fab, Fab′, F(ab′)₂ or scFv . . . ). In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four (α, δ, γ) to five (μ, ε) domains, a variable domain (VH) and three to four constant domains (CHI, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated VL-CDR1, VL-CDR2, VL-CDR3 and VH-CDR1, VH-CDR2, VH-CDR3, respectively. An antigen-binding site, therefore, typically includes six CDRs, comprising the CDR set from each of a heavy and a light chain V region. Framework Regions (FRs) refer to amino acid sequences interposed between CDRs. According to the invention, the amino acid residues in the variable domain, complementarity determining regions (CDRs) and framework regions (FR) of the antibody of the present invention are identified using the Immunogenetics (IMGT) database (http://imgt.cines.fr). Lefranc et al. (2003) Dev Comp Immunol. 27(1):55-77. The IMGT database was developed using sequence information for immunoglobulins (IgGs), T-cell receptors (TcR) and Major Histocompatibility Complex (MHC) molecules and unifies numbering across antibody lambda and kappa light chains, heavy chains and T-cell receptor chains and avoids the use of insertion codes for all but uncommonly long insertions. IMGT also takes into account and combines the definition of the framework (FR) and complementarity determining regions (CDR) from Kabat et al., the characterization of the hypervariable loops from Chothia et al., as well as structural data from X-ray diffraction studies.

The term “variable” refers to the fact that certain segments of the V domains differ extensively in sequence among antibodies. The V domain mediates antigen binding and defines specificity of a particular antibody for its particular antigen. However, the variability is not evenly distributed across the 110 to 130-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a [beta]-sheet configuration, connected by three hypervariable regions, which form loops connecting, and in some cases forming part of, the [beta]-sheet structure. The hypervariable regions in each chain are held together in close proximity by the FRs and, with the hypervariable regions from the other chain, contribute to the formation of the antigen-binding site of antibodies.

The “variable domain” or “variable region” of an antibody refers to the amino-terminal domains of the heavy or light chain of the antibody. The variable domain of the heavy chain may be referred to as “VH”. The variable domain of the light chain may be referred to as “VL”. These domains are generally the most variable parts of an antibody and contain the antigen-binding sites.

The term “hypervariable region” when used herein refers to the amino acid residues of an antibody that are responsible for antigen binding. The hypervariable region generally comprises amino acid residues from a “complementarity determining region” or “CDR”.

A “nanobody” is well known in the art and refers to an antibody-derived therapeutic protein that contains the unique structural and functional properties of naturally-occurring heavy chain antibodies. These heavy chain antibodies contain a single variable domain (VHH) and two constant domains (CH2 and CH3). As used herein, the term “derived” indicates a relationship between a first and a second molecule. It generally refers to structural similarity between the first molecule and the second molecule and does not connote or include a process or source limitation on a first molecule that is derived from a second molecule.

The terms “monoclonal antibody”, “monoclonal Ab”, “monoclonal antibody composition”, “mAb”, or the like, as used herein refer to a preparation of antibody molecules of single molecular composition. A monoclonal antibody is obtained from a population of substantially homogeneous antibodies, i.e., the individual antibodies comprised in the population are identical except for possible naturally occurring mutations that may be present in minor amounts. A monoclonal antibody composition displays a single binding specificity and affinity for a particular epitope. For instance monoclonal antibodies may be generated using the method of Kohler and Milstein (Nature, 256:495, 1975). To prepare monoclonal antibodies useful in the invention, a mouse or other appropriate host animal can be immunized at suitable intervals (e.g., twice-weekly, weekly, twice-monthly or monthly) with the appropriate antigenic forms (i.e., CD160-TM polypeptides). The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes. Monoclonal antibodies useful in the invention can also be prepared from non immunized animals or humans. However, the modifier “monoclonal” is not to be construed as requiring production of the antibody by any particular method. For example, a monoclonal antibody may also be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). A “monoclonal antibody” may also be isolated from phage antibody libraries using the techniques described in Clackson et al., Nature, 352:624-628 (1991) and Marks et al., J. Mol. Biol., 222:581-597 (1991), for example.

As used herein, the term “chimeric antibody” refers to an antibody which comprises a VH domain and a VL domain of a non-human antibody, and a CH domain and a CL domain of a human antibody.

The term “humanized antibody” refers to an antibody having variable region framework and constant regions from a human antibody but retains the CDRs of a previous non-human antibody. In one embodiment, a humanized antibody contains minimal sequence derived from non-human immunoglobulin. For the most part, humanized antibodies and antibody fragments thereof may be human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity.

As used herein the term “human monoclonal antibody”, is intended to include antibodies having variable and constant regions derived from human immunoglobulin sequences. The human antibodies of the present invention may include amino acid residues not encoded by human immunoglobulin sequences (e.g., mutations introduced by random or site-specific mutagenesis in vitro or by somatic mutation in vivo). More specifically, the term “human monoclonal antibody”, as used herein, is not intended to include antibodies in which CDR sequences derived from the germline of another mammalian species, such as a mouse, have been grafted onto human framework sequences.

As used herein, the term “epitope” refers to a specific arrangement of amino acids located on a protein or proteins to which an antibody binds. Epitopes often consist of a chemically active surface grouping of molecules such as amino acids or sugar side chains, and have specific three dimensional structural characteristics as well as specific charge characteristics. Epitopes can be linear or conformational, i.e., involving two or more sequences of amino acids in various regions of the antigen that may not necessarily be contiguous.

As used herein, the term “B cell,” refers to a type of lymphocyte in the humoral immunity of the adaptive immune system. B cells principally function to make antibodies, serve as antigen presenting cells, release cytokines, and develop memory B cells after activation by antigen interaction. B cells are distinguished from other lymphocytes by the presence of a B-cell receptor (i.e. an antibody) on the cell surface.

The term “nuclease” as used herein includes a protein (i.e. an enzyme) that induces a break in a nucleic acid sequence, e.g., a single or a double strand break in a double-stranded DNA sequence.

As used herein, the term “CRISPR/Cas nuclease” has its general meaning in the art and refers to segments of prokaryotic DNA containing clustered regularly interspaced short palindromic repeats (CRISPR) and associated nucleases encoded by Cas genes. In bacteria the CRISPR/Cas loci encode RNA-guided adaptive immune systems against mobile genetic elements (viruses, transposable elements and conjugative plasmids). Three types of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR/Cas nucleases Cas9 and Cpf1 belong to the type II and type V CRISPR/Cas system and have strong endonuclease activity to cut target DNA. Cas9 is guided by a mature crRNA that contains about 20 nucleotides of unique target sequence (called spacer) and a trans-activating small RNA (tracrRNA) that also serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG for S. pyogenes Cas9) protospacer adjacent motif (PAM) to specify the cut site (the 3^{rd} or the 4^{th} nucleotide upstream from PAM).

As used herein, the term “defective CRISPR/Cas nuclease” refers to a CRISPR/Cas nuclease having lost its nuclease activity.

As used herein, the term “guide RNA” generally refers to an RNA molecule (or a group of RNA molecules collectively) that can bind to a CRISPR protein and target the CRISPR protein to a specific location within a target DNA. A guide RNA can comprise two segments: a DNA-targeting guide segment and a protein-binding segment. The DNA-targeting segment comprises a nucleotide sequence that is complementary to (or at least can hybridize to under stringent conditions) a target sequence. The protein-binding segment interacts with a CRISPR protein, such as a Cas9 or Cas9 related polypeptide. These two segments can be located in the same RNA molecule or in two or more separate RNA molecules. When the two segments are in separate RNA molecules, the molecule comprising the DNA-targeting guide segment is sometimes referred to as the CRISPR RNA (crRNA), while the molecule comprising the protein-binding segment is referred to as the trans-activating RNA (tracrRNA).

As used herein, the term “target nucleic acid” or “target” refers to a nucleic acid containing a target nucleic acid sequence. A target nucleic acid may be single-stranded or double-stranded, and often is double-stranded DNA. A “target nucleic acid sequence,” “target sequence” or “target region,” as used herein, means a specific sequence or the complement thereof that one wishes to bind to using the CRISPR system as disclosed herein. According to the present invention target sequence is within a nucleic acid that encodes for a binding domain of the binding protein (e.g. a variable domain of an antibody).

A “target nucleic acid strand” refers to a strand of a target nucleic acid that is subject to base-pairing with a guide RNA as disclosed herein. That is, the strand of a target nucleic acid that hybridizes with the crRNA and guide sequence is referred to as the “target nucleic acid strand.” The other strand of the target nucleic acid, which is not complementary to the guide sequence, is referred to as the “non-complementary strand.” In the case of double-stranded target nucleic acid (e.g., DNA), each strand can be a “target nucleic acid strand” to design crRNA and guide RNAs and used to practice the method of this invention as long as there is a suitable PAM site.

As used herein, the term “non-nuclease DNA modifying enzyme” refers to an enzyme that is not a nuclease but can introduce some modifications in a DNA molecule, such as a mutation.

The term “cytidine deaminase” refers to enzyme that catalyzes the irreversible hydrolytic deamination of cytidine and deoxycytidine to uridine and deoxyuridine, respectively. The term “deamination”, as used herein, refers to the removal of an amine group from one molecule.

The term “AID” or “Activation-Induced cytidine deaminase” refers to an enzyme that belongs to the APOBEC family of cytidine deaminase enzymes. AID is expressed within activated B cells and is required to initiate somatic hypermutation (Muramatsu et al., Cell, 102(5): 553-63 (2000); Revy et al., Cell, 102(5): 565-75 (2000); Yoshikawa et al., Science, 296(5575): 2033-6 (2002)) by creating point mutations in the underlying DNA encoding antibody genes (Martin et al., Proc. Natl. Acad. Sci. USA., 99(19): 12304-12308 (2002) and Nature, 415(6873): (2002); Petersen-Mart et al., Nature, 418(6893): 99-103 (2002)). AID is also an essential protein factor for class switch recombination and gene conversion (Muramatsu et al., Cell, 102(5): 553-63 (2000); Revy et al., Cell, 102(5): 565-75 (2000)).

As used herein, the term “ribonucleoprotein complex,” or “ribonucleoprotein particle” refers to a complex or particle including a nucleoprotein and a ribonucleic acid. A “nucleoprotein” as provided herein refers to a protein capable of binding a nucleic acid (e.g., RNA, DNA). Where the nucleoprotein binds a ribonucleic acid it is referred to as “ribonucleoprotein.” The interaction between the ribonucleoprotein and the ribonucleic acid may be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

As used herein the term “wild type” is a term of the art understood by skilled persons and means the typical form of an organism, strain, gene or characteristic as it occurs in nature as distinguished from mutant or variant forms.

As used herein, the term “mutation” has its general meaning in the art and refers to a substitution, deletion or insertion. The term “substitution” means that a specific amino acid residue at a specific position is removed and another amino acid residue is inserted into the same position. The term “deletion” means that a specific amino acid residue is removed. The term “insertion” means that one or more amino acid residues are inserted before or after a specific amino acid residue.

As used herein, the term “mutagenesis” refers to the introduction of mutations into a polynucleotide sequence. According to the present invention mutations are introduced into a target DNA molecule encoding for a variant domain of the antibody so as to mimic somatic hypermutation.

As used herein, the term ‘somatic hypermutation” has its general meaning in the art and refers to the phenomenon in which a high frequency of point mutations are generated within a 1-2-kb segment in the variable region of expressed immunoglobulin genes in response to the presence of an antigen.

As used herein, the term “variant” refers to a first composition (e.g., a first molecule), that is related to a second composition (e.g., a second molecule, also termed a “parent” molecule). The variant molecule can be derived from, isolated from, based on or homologous to the parent molecule. A variant molecule can have entire sequence identity with the original parent molecule, or alternatively, can have less than 100% sequence identity with the parent molecule. For example, a variant of a sequence can be a second sequence that is at least 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100% identical in sequence compare to the original sequence. Sequence identity is frequently measured in terms of percentage identity (or similarity or homology); the higher the percentage, the more similar are the two sequences. Methods of alignment of sequences for comparison are well known in the art. Various programs and alignment algorithms are described in: Smith and Waterman, Adv. Appl. Math., 2:482, 1981; Needleman and Wunsch, J. Mol. Biol., 48:443, 1970; Pearson and Lipman, Proc. Natl. Acad. Sci. U.S.A., 85:2444, 1988; Higgins and Sharp, Gene, 73:237-244, 1988; Higgins and Sharp, CABIOS, 5:151-153, 1989; Corpet et al. Nuc. Acids Res., 16:10881-10890, 1988; Huang et al., Comp. Appls Biosci., 8:155-165, 1992; and Pearson et al., Meth. Mol. Biol., 24:307-31, 1994). Altschul et al., Nat. Genet., 6:119-129, 1994, presents a detailed consideration of sequence alignment methods and homology calculations. By way of example, the alignment tools ALIGN (Myers and Miller, CABIOS 4:11-17, 1989) or LFASTA (Pearson and Lipman, 1988) may be used to perform sequence comparisons (Internet Program® 1996, W. R. Pearson and the University of Virginia, fasta20u63 version 2.0u63, release date December 1996). ALIGN compares entire sequences against one another, while LFASTA compares regions of local similarity. These alignment tools and their respective tutorials are available on the Internet at the NCSA Website, for instance. Alternatively, for comparisons of amino acid sequences of greater than about 30 amino acids, the Blast 2 sequences function can be employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). The BLAST sequence comparison system is available, for instance, from the NCBI web site; see also Altschul et al., J. Mol. Biol., 215:403-410, 1990; Gish. & States, Nature Genet., 3:266-272, 1993; Madden et al. Meth. Enzymol., 266:131-141, 1996; Altschul et al., Nucleic Acids Res., 25:3389-3402, 1997; and Zhang & Madden, Genome Res., 7:649-656, 1997.

As used herein, the term “Major Histocompatibility Complex” (MHC) is a generic designation meant to encompass the histo-compatibility antigen systems described in different species including the human leucocyte antigens (HLA). In humans there are three major different genetic loci that encode MHC class I molecules (the MHC-molecules of the human are also designated human leukocyte antigens (HLA)): HLA-A, HLA-B, and HLA-C. HLA-A*01, HLA-A*02, and HLA-A*11 are examples of different MHC class I alleles that can be expressed from these loci. It should be further noted that nonclassical human MHC class I molecules such as HLA-E (functional homolog in mice is called Qa-1b) and MICA/B molecules are also encompassed within the context of the invention.

As used herein, the term “peptide MHC monomer” refers to a stable complex composed of major histocompatibility complex (MHC) protein subunits loaded with a the epitope peptide recognized by the antibody of interest.

The term “multimer” refers to a compound that comprises at least two target molecules coupled together, such as through a linker covalently bonded to the target molecules. Typically, the multimer comprises 2, 3, 4, 5, 6, 7, 8, 9 or 10 target molecules.

As used herein, the term “tetramer” may refer to a multimer comprising four target molecules. Typically the tetramer consist of 4 target molecules bound to a single molecule of streptavidin, which can bind to and thus identify a population of cells. A subunit can be a MHC-peptide complex loaded with a peptide epitope that can be recognized by an antibody. A population of cells identified by the tetramer can be a population that expresses the binding protein (e.g. the antibody) at the cell surface.

As used herein, the term “label” or “tag” refers to a molecule that can be bound by a binding molecule. Said label thus provides a detectable signal indicative of the presence of the complex to which the label is conjugated and also the immunomagnetic enrichment of the complex with nanoparticles to which binding molecules are attached.

As used herein, the term “magnetic particle” refers to a nano- or micro-scale particle that is attracted or repelled by a magnetic field gradient or has a non-zero magnetic susceptibility. The term “magnetic particle” also includes magnetic particles that have been conjugated with affinity molecules.

As used herein, the term “immunomagnetic enrichment” refers to procedures for cell separation (cell sorting) including magnetic separation using antibodies linked to magnetic particles.

As used herein, the term “binding molecule” or “affinity molecule” refers to any molecule that is capable of specifically binding a target component.

As used herein, the term “aptamer” means a single-stranded, partially single-stranded, partially double-stranded or double-stranded nucleotide sequence capable of specifically recognizing a selected non-oligonucleotide molecule or group of molecules by a mechanism other than Watson-Crick base pairing or triplex formation.

As used herein, the term “derived from” refers to a process whereby a first component (e.g., a first molecule), or information from that first component, is used to isolate, derive or make a different second component (e.g., a second molecule that is different from the first).

The term “fusion polypeptide” or “fusion protein” means a protein created by joining two or more polypeptide sequences together. The fusion polypeptides encompassed in this invention include translation products of a chimeric gene construct that joins the nucleic acid sequences encoding a first polypeptide, e.g., an RNA-binding domain, with the nucleic acid sequence encoding a second polypeptide, e.g., an effector domain, to form a single open-reading frame. In other words, a “fusion polypeptide” or “fusion protein” is a recombinant protein of two or more proteins which are joined by a peptide bond or via several peptides. The fusion protein may also comprise a peptide linker between the two domains.

The term “linker” refers to any means, entity or moiety used to join two or more entities. A linker can be a covalent linker or a non-covalent linker. Examples of covalent linkers include covalent bonds or a linker moiety covalently attached to one or more of the proteins or domains to be linked. The linker can also be a non-covalent bond, e.g., an organometallic bond through a metal center such as platinum atom. For covalent linkages, various functionalities can be used, such as amide groups, including carbonic acid derivatives, ethers, esters, including organic and inorganic esters, amino, urethane, urea and the like. To provide for linking, the domains can be modified by oxidation, hydroxylation, substitution, reduction etc. to provide a site for coupling. Methods for conjugation are well known by persons skilled in the art and are encompassed for use in the present invention. Linker moieties include, but are not limited to, chemical linker moieties, or for example a peptide linker moiety (a linker sequence). It will be appreciated that modification which do not significantly decrease the function of the RNA-binding domain and effector domain are preferred.

As used herein, the term “conjugate” or “conjugation” or “linked” as used herein refers to the attachment of two or more entities to form one entity. A conjugate encompasses both peptide-small molecule conjugates as well as peptide-protein/peptide conjugates.

As used herein, the term “sequencing” generally means a process for determining the order of nucleotides in a nucleic acid. A variety of methods for sequencing nucleic acids is well known in the art and can be used.

As used herein, the term “next generation sequencing” has its general meaning in the art and refers to sequencing technologies having increased throughput as compared to traditional Sanger- and capillary electrophoresis-based approaches, for example with the ability to generate hundreds of thousands or millions of relatively short sequence reads at a time.

As used herein, the term “contacting,” when used in reference to any set of components, includes any process whereby the components to be contacted are mixed into same mixture (for example, are added into the same compartment or solution), and does not necessarily require actual physical contact between the recited components. The recited components can be contacted in any order or any combination (or sub-combination), and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C. “Contacting” a target nucleic acid or a cell with one or more reaction components, such as an CRISPR/Cas nuclease or guide RNA, includes any or all of the following situations: (i) the target or cell is contacted with a first component of a reaction mixture to create a mixture; then other components of the reaction mixture are added in any order or combination to the mixture; and (ii) the reaction mixture is fully formed prior to mixture with the target or cell.

The term “mixture” as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not spatially distinct. In other words, a mixture is not addressable.

### Methods

The first object of the present invention relates to a method of generating and selecting a variant of a binding protein with increased binding affinity and/or specificity that consists of subjecting a population of cells that express the binding protein to at least one round of mutagenesis coupled to an affinity/specificity-based selection and immunomagnetic enrichment.

Population of Cells:

The population of cells encompasses any population of cells that can express the binding protein preferably at the cell surface.

In some embodiments, the population of cells express an antibody (i.e. a monoclonal antibody) at the cell surface.

In some embodiments, the population of cells is a population of B cells. In some embodiments, the population of cells is not a population of B cells, but a population of eukaryotic cell engineered for expressing the light and heavy chains of the antibody of interest. Typically, the cells are HEK 293 cells engineered to express at the cell surface the antibody by stable transfection of nucleic acid molecules for encoding the light and heavy chain of the antibody.

In some embodiments, the antibody that is expressed by the population of cells invention is a whole antibody, i.e. an antibody having 2 light chains and 2 heavy chains. In some embodiments, the antibody is selected from the following group: a whole antibody, a human antibody, a humanized antibody, a single chain antibody, a dimeric single chain antibody, a Fv, a scFv, a Fab, a F(ab)′₂, a bi-specific antibody, a diabody, a triabody, a tetrabody. In some embodiments, said antibody is an antibody fragment selected from a group consisting of a unibody, a domain antibody, and a nanobody.

Step (A): Mutagenesis:

The mutagenesis consists of contacting the population of cells engineered for expressing the binding protein with a gene editing platform that consists of a (a) a defective CRISPR/Cas nuclease engineered for sequence targeting, (b) a non-nuclease DNA modifying enzyme and (c) a plurality of RNA molecules for guiding the defective nuclease and the non-nuclease RNA modifying enzyme to a plurality of target sequences in the DNA nucleic acid molecule coding for the binding domain of the binding protein (e.g. a variable domain of an antibody).

According to the present invention the cells and the gene editing platform are contacted for a time sufficient for allowing the introduction of mutations in the DNA nucleic acid molecule coding for the binding domain of the binding protein. In some embodiments, the cells and the gene editing platform are contacted with the gene editing platform for a plurality of times (e.g. 2, 3, 4, 5, 6 times).

Illustrated in FIG. 2 is a schematic of one embodiment of the mutagenesis process that is carried for an antibody. More specifically, the system includes three structural and functional components summarized in FIG. 2A: (a) a defective CRISPR/Cas nuclease engineered for sequence targeting (i.e. a dCas9 protein); (b) a non-nuclease DNA modifying enzyme (i.e. MS2 coat protein fused to AID*D) and (c) a plurality of RNA molecules for guiding the defective nuclease and the non-nuclease RNA modifying enzyme to a plurality of target sequences (see FIG. 2B) in the DNA nucleic acid molecule coding for a variable domain of an antibody (i.e. the guide RNAs linked to the MS2 hairpin sequences).

The different embodiments of these three structural and functional components are described hereinafter.

(a) The Defective CRISPR/Cas Nuclease Engineered for Sequence Targeting

According to the present invention, the sequence targeting component of the above system involves use of a defective CRISPR/Cas nuclease. The sequence recognition mechanism is the same as for the non-defective CRISPR/Cas nuclease. Typically, the defective CRISPR/Cas nuclease of the invention comprises at least one RNA binding domain. The RNA binding domain interacts with a guide RNA as defined hereinafter. However the defective CRISPR/Cas nuclease of the invention is a modified version with no nuclease activity. Accordingly, the defective CRISPR/Cas nuclease specifically recognizes the guide RNA and thus guides the nuclease to its target DNA sequence.

In some embodiments, the defective CRISPR/Cas nuclease can be modified to increase nucleic acid binding affinity and/or specificity, alter an enzymatic activity, and/or change another property of the protein. In some embodiments, the nuclease domains of the protein can be modified, deleted, or inactivated. In some embodiments, the protein can be truncated to remove domains that are not essential for the function of the protein. In some embodiments, the protein is truncated or modified to optimize the activity of the RNA binding domain.

In some embodiments, the CRISPR/Cas nuclease consists of a mutant CRISPR/Cas nuclease i.e. a protein having one or more point mutations, insertions, deletions, truncations, a fusion protein, or a combination thereof. In some embodiments, the mutant has the RNA-guided DNA binding activity, but lacks one or both of its nuclease active sites. In some embodiments, the mutant comprises an amino acid sequence having at least 50% of identity with the wild type amino acid sequence of the CRISPR/Cas nuclease.

Various CRISPR/Cas nucleases can be used in this invention. Non-limiting examples of suitable CRISPR/CRISPR/Cas nucleases include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966. See e.g., WO2014144761 WO2014144592, WO2013176772, US20140273226, and US20140273233, the contents of which are incorporated herein by reference in their entireties.

In some embodiments, the CRISPR/Cas nuclease is derived from a type II CRISPR-Cas system. In some embodiments, the CRISPR/Cas nuclease is derived from a Cas9 protein. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina, inter alia.

In some embodiments, the CRISPR/Cas nuclease is a mutant of a wild type CRISPR/Cas nuclease (such as Cas9) or a fragment thereof. In some embodiments, the CRISPR/Cas nuclease is a mutant Cas9 protein from S. pyogenes. In some embodiments, the CRISPR/Cas nuclease of the present invention is a defective Cas9, i.e. the Cas9 from S. pyrogenes having at least one mutation selected from the group consisting of D10A and H840A. In some embodiments, the CRISPR/Cas nuclease of the present invention comprises the amino acid sequence as set forth in SEQ ID NO: 1.

> S. pyogenes dCas9 Protein Sequence
SEQ ID NO: 1
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

(b) The Non-Nuclease DNA Modifying Enzyme

The second component of the gene editing platform herein disclosed comprises a non-nuclease DNA modifying enzyme.

The DNA modifying enzyme is not a nuclease and does not have any nuclease activity, but can have the activity of other types of DNA modifying enzymes. Examples of the enzymatic activity include, but are not limited to, deamination activity, methyltransferase activity, demethylase activity, DNA repair activity, DNA damage activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity. In some embodiments, the DNA modifying enzyme induces somatic hypermutation (SMH).

In some embodiments, the DNA modifying enzyme has the activity of cytosine deaminases (e.g., AID, APOBEC3G), adenosine deaminases (e.g., ADA), DNA methyltransferases, and DNA demethylases.

In some embodiments, the DNA modifying enzyme is selected from the group consisting of AID: activation induced cytidine deaminase, APOBEC1: apolipoprotein B mRNA editing enzyme, catalytic polypeptide-like 1, APOBEC3A: apolipoprotein B mRNA editing enzyme, catalytic polypeptide-like 3A, APOBEC3B: apolipoprotein B mRNA editing enzyme, catalytic polypeptide-like 3B, APOBEC3C: apolipoprotein B mRNA editing enzyme, catalytic polypeptide-like 3C, APOBEC3D: apolipoprotein B mRNA editing enzyme, catalytic polypeptide-like 3D, APOBEC3F: apolipoprotein B mRNA editing enzyme, catalytic polypeptide-like 3F, APOBEC3G: apolipoprotein B mRNA editing enzyme, catalytic polypeptide-like 3G, APOBEC3H: apolipoprotein B mRNA editing enzyme, catalytic polypeptide-like 3H, ADA: adenosine deaminase, ADAR1: adenosine deaminase acting on RNA 1, Dnmt1: DNA (cytosine-5-)-methyltransferase 1, Dnmt3a: DNA (cytosine-5-)-methyltransferase 3 alpha, Dnmt3b: DNA (cytosine-5-)-methyltransferase 3 beta and Tet1: methylcytosine dioxygenase.

In some embodiments, the DNA modifying enzyme derives from the Activation Induced cytidine Deaminase (AID). AID is a cytidine deaminase that can catalyze the reaction of deamination of cytosine in the context of DNA or RNA. When brought to the targeted site, AID changes a C base to U base. In dividing cells, this could lead to a C to T point mutation. Alternatively, the change of C to U could trigger cellular DNA repair pathways, mainly excision repair pathway, which will remove the mismatching U-G base-pair, and replace with a T-A, A-T, C-G, or G-C pair. As a result, a point mutation would be generated at the target C-G site. In some embodiments, the DNA modifying enzyme is AID*Δ that is an AID mutant with increased SHM activity whose Nuclear Export Signal (NES) has been removed (Hess G T, Fresard L, Han K, Lee CH, Li A, Cimprich K A, Montgomery S B, Bassik M C: Directed evolution using dCas9-targeted somatic hypermutation in mammalian cells. Nat Methods 2016, 13(12): 1036-1042). In some embodiments, the AID*Δ has the amino acid sequence as set forth in SEQ ID NO:2.

> AID*Δ
SEQ ID NO: 2
MDSLLMNRREFLYQFKNVRWAKGRRETYLCYVVKRRDSATSFSLDFGYLR
NKNGCHVELLFLRYISDWDLDPGRCYRVTWFISWSPCYDCARHVADFLRG
NPNLSLRIFTARLYFCEDRKAEPEGLRRLHRAGVQIAIMTFKDYFYCWNT
FVENHGRTFKAWEGLHENSVRLSRQLRRILLPLYEVDDLRDAFRTCTGSG

In some embodiments, the second component is a fusion protein wherein the DNA modifying enzyme is fused to an RNA-binding domain. In some embodiments, the DNA modifying enzyme is fused directly or via a linker to the RNA binding domain. In some embodiments, the last amino acid of the C-terminal end of the DNA modifying enzyme is directly linked by a covalent bond to the first amino acid of the N-terminal end of said RNA binding domain, or the first amino acid of the N-terminal end of said polypeptide is directly linked by a covalent bond to the last amino acid of the C-terminal end of said RNA binding domain. In some embodiments, the linker is a peptidic linker which comprises at least one, but less than 30 amino acids e.g., a peptidic linker of 2-30 amino acids, preferably of 10-30 amino acids, more preferably of 15-30 amino acids, still more preferably of 19-27 amino acids, most preferably of 20-26 amino acids. In some embodiments, the linker has 2; 3; 4; 5; 6; 7; 8; 9; 10; 11; 12; 13; 14; 15; 16; 17; 18; 19; 20; 21; 22; 23; 24; 25; 26; 27; 28; 29; 30 amino acid residues. Typically, linkers are those which allow the compound to adopt a proper conformation. The most suitable linker sequences (1) will adopt a flexible extended conformation, (2) will not exhibit a propensity for developing ordered secondary structure which could interact with the functional domains of fusion proteins, and (3) will have minimal hydrophobic or charged character which could promote interaction with the functional protein domains. Typical surface amino acids in flexible protein regions include Gly, Asn and Ser (i.e., G, N or S). Virtually any permutation of amino acid sequences containing Gly, Asn and Ser would be expected to satisfy the above criteria for a linker sequence. Other near neutral amino acids, such as Thr, Ala, Leu, Gln (i.e., T, A, L, Q) may also be used in the linker sequence. The length of the linker sequence may vary without significantly affecting the biological activity of the fusion protein. Exemplary linker sequences are described in U.S. Pat. Nos. 5,073,627 and 5,108,910. In some examples, the linker is a immunoglobulin hinge region linker as disclosed in U.S. Pat. Nos. 6,165,476, 5,856,456, US Application Nos. 20150182596 and 2010/0063258 and International Application WO2012/142515, each of which are incorporated herein in their entirety by reference.

Although various RNA-binding domains can be used in this invention, the RNA-binding domain of CRISPR/Cas nuclease (such as Cas9) or its variant (such as dCas9) should not be used. As mentioned above, the direct fusion to dCas9, which anchors to DNA in a defined conformation, would hinder the formation of a functional oligomeric enzyme complex at the right location. Instead, the present invention takes advantages of various other RNA motif-RNA binding protein binding pairs. In this way, the DNA modifying enzyme can be recruited to the target site through RNA-binding domain's ability to bind to the recruiting RNA motif. Due to the flexibility of the RNA molecule (i.e. third component of the gene editing platform of the present invention) mediated recruitment, a functional monomer, as well as dimer, tetramer, or oligomer could be formed relatively easily near the target DNA sequence.

In some embodiments, the RNA binding domain derives from a protein selected from the group consisting of the telomerase Sm7, MS2 Coat Protein, PP7 coat protein (PCP), SfMu phage Com RNA binding protein. In some embodiments, the RNA binding domain is an aptamer. In some embodiments, the RNA binding domain is the MS2 coat protein variant having an amino acid sequence as set forth in SEQ ID NO:3.

> MS2 coat protein variant
SEQ ID NO: 3
MASNFTQFVLVDNGGYGDVTVAPSNFANGIAEWISSNSRSQAYKVTCSVR
QSSAQNRKYTIKVEVPKGAWRSYLNMELTIPIFATNSDCELIVKAMQGLL
KDGNPIPSAIAANSGIY

Like the CRISPR/Cas nuclease described above, the non-nuclease DNA modifying enzyme can also be obtained as a recombinant polypeptide. Techniques for making recombinant polypeptides are known in the art. See e.g., Creighton, “Proteins: Structures and Molecular Principles,” W.H. Freeman & Co., NY, 1983); Ausubel et al., Current Protocols in Molecular Biology, John Wiley & Sons, 2003; and Sambrook et al., Molecular Cloning, A Laboratory Manual,” Cold Spring Harbor Press, Cold Spring Harbor, N.Y., 2001).

(c) RNA Molecules for Guiding the Defective Nuclease and the Non-Nuclease RNA Modifying Enzyme

The third component of the platform disclosed herein consists of a plurality of RNA molecules suitable for guiding the defective nuclease and the non-nuclease RNA modifying enzyme to a plurality of target sequences in the DNA nucleic acid encoding for the binding domain of the binding protein (e.g. a variable domain of an antibody).

Typically, the RNA molecule of the present invention has three sub-components: a programmable guide RNA motif, a CRISPR RNA motif, and a recruiting RNA motif. As disclosed herein, the programmable guide RNA, CRISPR RNA and the CRISPR/Cas nuclease together form a CRISPR/Cas-based module for sequence targeting and recognition, while the recruiting RNA motif via an RNA-protein binding pair recruits the non-nuclease DNA modifying enzyme suitable for introducing a plurality of mutation in the DNA nucleic acid molecule encoding for the binding domain of the binding protein (e.g. a variable domain of an antibody) (see FIG. 2A). Accordingly, the RNA molecule connects the CRISPR/Cas nuclease and the the non-nuclease DNA modifying enzyme.

The first sub-component of the RNA molecule of the present invention comprises a guide sequence for providing the targeting specificity. It includes a region that is complementary and capable of hybridization to a pre-selected target site of interest. In some embodiment, this guide sequence can comprise from about 10 nucleotides to more than about 25 nucleotides. For example, the region of base pairing between the guide sequence and the corresponding target site sequence can be about 10, 11, 12, 13, 14, 15, 16, 17, 18, 19, 20, 22, 23, 24, 25, or more than 25 nucleotides in length. In some embodiments, the guide sequence is about 17-20 nucleotides in length, such as 20 nucleotides.

Typically, a software program is used to identify candidate CRISPR target sequences on both strands of the DNA nucleic acid molecule encoding for a binding domain of the binding protein (e.g. a variable domain of an antibody), based on desired guide sequence length and a CRISPR motif sequence (PAM) for a specified CRISPR enzyme. One requirement for selecting a suitable target nucleic acid is that it has a 3′ PAM site/sequence. Each target sequence and its corresponding PAM site/sequence are referred herein as a Cas-targeted site. Type II CRISPR system, one of the most well characterized systems, needs only Cas 9 protein and a guide RNA complementary to a target sequence to affect target cleavage. For example, target sites for Cas9 from S. pyogenes, with PAM sequences NGG, may be identified by searching for 5′-Nx-NGG-3′ both on the input sequence and on the reverse-complement of the input. Since multiple occurrences in the genome of the DNA target site may lead to nonspecific genome editing, after identifying all potential sites, the program filters out sequences based on the number of times they appear in the relevant reference genome. For those CRISPR enzymes for which sequence specificity is determined by a “seed” sequence, such as the 11-12 bp 5′ from the PAM sequence, including the PAM sequence itself, the filtering step may be based on the seed sequence. Thus, to avoid editing at additional genomic loci, results are filtered based on the number of occurrences of the seed:PAM sequence in the relevant genome. The user may be allowed to choose the length of the seed sequence. The user may also be allowed to specify the number of occurrences of the seed:PAM sequence in a genome for purposes of passing the filter. The default is to screen for unique sequences. Filtration level is altered by changing both the length of the seed sequence and the number of occurrences of the sequence in the genome. The program may in addition or alternatively provide the sequence of a guide sequence complementary to the reported target sequence(s) by providing the reverse complement of the identified target sequence(s). Further details of methods and algorithms to optimize sequence selection can be found in U.S. application Ser. No. 61/836,080; incorporated herein by reference.

According to the present invention, a plurality of RNA molecules are designed for targeting a plurality of sequences in the DNA nucleic acid molecule encoding for a binding domain of the binding protein (e.g. a variable domain of an antibody) (see FIG. 2B). In some embodiments, the third component of the platform disclosed herein thus comprises 2, 3, 4, 5, 6, 7, 8, 9, 10, 11, 12, 13, 14, 15, 16, 17, 18, or 20 RNA molecules as disclosed herein.

The second sub-component of the RNA molecule of the present invention includes additional active or non-active sub-components. In some embodiments, the RNA molecule has a CRISPR motif with tracrRNA activity. In some embodiments, the RNA molecule is a hybrid RNA molecule where the above-described programmable guide RNA is fused to a tracrRNA to mimic the natural crRNA:tracrRNA duplex (Chen et al. Cell. 2013 Dec. 19; 155(7):1479-91). Various tracrRNA sequences are known in the art and examples include the following tracrRNAs and active portions thereof. As used herein, an active portion of a tracrRNA retains the ability to form a complex with a CRISPR/Cas nuclease, such as Cas9 or dCas9. See, e.g., WO2014144592. Methods for generating crRNA-tracrRNA hybrid RNAs are known in the art. See e.g., WO2014099750, US 20140179006, and US 20140273226. The contents of these documents are incorporated herein by reference in their entireties. In some embodiments, the tracrRNA activity and the guide sequence are two separate RNA molecules, which together form the guide RNA and related scaffold. In this case, the molecule with the tracrRNA activity should be able to interact with (usually by base pairing) the molecule having the guide sequence.

The third sub-component of the RNA molecule is the recruiting RNA motif that is suitable for linking the first (i.e. the defective CRISPR/Cas nuclease) and the second component (i.e. the non-nuclease DNA modifying enzyme) of the gene editing platform. The present invention takes advantages of various RNA motif/RNA binding protein binding pairs. To this end, the RNA molecule comprises at least one RNA sequence (e.g., MS2 hairpin as depicted in FIG. 2A) that specifically binds to the RNA binding protein (e.g., MS2 coat protein as depicted in FIG. 2B) fused to the non-nuclease DNA modifying enzyme as described above. As a result, the RNA molecule of the gene editing platform disclosed herein is a designed RNA molecule, which contains not only the gRNA motif for specific DNA/RNA sequence recognition, the CRISPR RNA motif for dCas9 binding, but also at least one recruiting RNA motif for recruiting the DNA modifying enzyme. In this way, the DNA modifying enzyme can be recruited to the target site through their ability to bind to the recruiting RNA motif. In some embodiments, the RNA molecule of the present invention comprises 2, 3, 4, 5, 6, 7, 8, 9 or 10 recruiting RNA motif (e.g. see FIG. 2A).

The pairs of RNA recruiting motif/binding protein could be derived from naturally occurring sources (e.g., RNA phages, or yeast telomerase) or could be artificially designed (e.g., RNA aptamers and their corresponding binding protein ligands). A non-exhausting list of examples of recruiting RNA motif/RNA binding protein pairs that could be used in the present invention are the telomerase Ku binding motif, the telomerase Sm7 binding motif, the MS2 phage operator stem-loop, the PP7 phage operator stem-loop, or the SfMu phage Com stem-loop. In some embodiments, the RNA recruiting motif comprise the MS2 Phage Operator Stem Loop as set forth in SEQ ID NO:5.

> MS2 Phage Operator Stem Loop
SEQ ID NO: 4
5′-GCGCACATGAGGATCACCCATGTGC-3′

The RNA molecule of the present invention can be made by various methods known in the art including cell-based expression, in vitro transcription, and chemical synthesis. The ability to chemically synthesize relatively long RNAs (as long as 200 mers or more) using TC-RNA chemistry (see, e.g., U.S. Pat. No. 8,202,983) allows one to produce RNAs with special features that outperform those enabled by the basic four ribonucleotides (A, C, G and U). In particular, the RNA molecule of the present invention can be made with recombinant technology using a host cell system or an in vitro translation-transcription system known in the art. Details of such systems and technology can be found in e.g., WO2014144761 WO2014144592, WO2013176772, US20140273226, and US20140273233, the contents of which are incorporated herein by reference in their entireties.

The RNA molecule may include one or more modifications. Such modifications may include inclusion of at least one non-naturally occurring nucleotide, or a modified nucleotide, or analogs thereof. Modified nucleotides may be modified at the ribose, phosphate, and/or base moiety. Modified nucleotides may include 2′-O-methyl analogs, 2′-deoxy analogs, or 2′-fluoro analogs. The nucleic acid backbone may be modified, for example, a phosphorothioate backbone may be used. The use of locked nucleic acids (LNA) or bridged nucleic acids (BNA) may also be possible. Further examples of modified bases include, but are not limited to, 2-aminopurine, 5-bromo-uridine, pseudouridine, inosine, 7-methylguanosine.

In some embodiments, the different components of the gene editing platform of the present invention are provided to the population of cells through expression from one or more expression vectors. For example, the nucleic acids encoding the RNA molecule or proteins (CRISPR/cas endonuclease and the non-nuclease DNA modifying enzyme) can be cloned into one or more vectors for introducing them into the population of cells that express the binding protein. The vectors are typically prokaryotic vectors, e.g., plasmids, or shuttle vectors, or insect vectors, for storage or manipulation of the nucleic acid encoding the RNA molecule or protein for production of the RNA molecule or protein. Preferably, the nucleic acids are isolated and/or purified. Thus, the present invention provides recombinant constructs or vectors having sequences encoding one or more of the RNA molecule or proteins described above. Examples of the constructs include a vector, such as a plasmid or viral vector, into which a nucleic acid sequence of the invention has been inserted, in a forward or reverse orientation. In some embodiments, the construct further includes regulatory sequences. A “regulatory sequence” includes promoters, enhancers, and other expression control elements (e.g., polyadenylation signals). Regulatory sequences include those that direct constitutive expression of a nucleotide sequence, as well as inducible regulatory sequences. The design of the expression vector can depend on such factors as the choice of the host cell to be transformed, transfected, or infected, the level of expression of RNAs or proteins desired, and the like. Large numbers of suitable vectors and promoters are known to those of skill in the art, and are commercially available. Appropriate cloning and expression vectors for use with eukaryotic hosts are also described in e.g., Sambrook et al. (2001, Molecular Cloning: A Laboratory Manual, Cold Spring Harbor Press). The vector can be capable of autonomous replication or integration into a host DNA. The vector may also include appropriate sequences for amplifying expression. In addition, the expression vector preferably contains one or more selectable marker genes to provide a phenotypic trait for selection of transformed host cells such as dihydrofolate reductase or neomycin resistance for eukaryotic cell cultures, or such as tetracycline or ampicillin resistance in E. coli. Any of the procedures known in the art for introducing foreign nucleotide sequences into host cells may be used. Examples include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell.

In some embodiments, the different components of the gene editing platform of the present invention are provided to the population of cells through the use of ribonucleoprotein (RNP) complexes. For instance the CRISPR/cas endonuclease and the the non-nuclease DNA modifying enzyme can be pre-complexed with the plurality of RNA molecules to form a ribonucleoprotein (RNP) complex. The RNP complex can thus be introduced into the population of cells. Introduction of the RNP complex can be timed. The celld can be synchronized with other cells at G1, S, and/or M phases of the cell cycle. RNP delivery avoids many of the pitfalls associated with mRNA, DNA, or viral delivery. Typically, the RNP complex is produced simply by mixing the proteins (i.e. the CRISPR/cas endonuclease and the the non-nuclease DNA modifying enzyme) and one or more RNA molecule in an appropriate buffer. This mixture is incubated for 5-10 min at room temperature before electroporation. Electroporation is a delivery technique in which an electrical field is applied to one or more cells in order to increase the permeability of the cell membrane. In some embodiments, genome editing efficiency can be improved by adding a transfection enhancer oligonucleotide.

In some embodiments, a plurality of successive transfection are performed before the cells are screened for expression of mutant antibodies with increased affinity.

B) Affinity/Specificity-Based Cell Selection and Immune Magnetic Enrichment

The affinity/specificity-based cell selection and immune magnetic enrichment starts with a step consisting in contacting the post-mutagenesis population of cells with a plurality of multimers made up by mixing specific and unspecific target molecules for the binding protein.

In some embodiments, the multimers are tetramers. In some embodiments, when the binding protein is an antibody, the epitope peptide that is specifically recognized by the antibody is loaded in a soluble peptide MHC monomer that is then tetramerized with three other soluble peptide MHC monomers that includes at least one non-specific MHC peptide monomer. Typically, the non-specific peptide monomer of a MHC molecule loaded with an irrelevant peptide (i.e. a peptide that is not recognized by the antibody of interest). In some embodiments, the tetramer thus comprises 4, 3, 2 or 1 specific MHC peptide monomer(s). In some embodiments, the MHC molecule is mutated, in particular in the α3 domain (A245V) for reducing CD8 binding to MHC class I molecules. Methods for obtaining tetramers are described in WO96/26962 and WO01/18053, which are incorporated by reference. In some embodiments, the tetramer may be a multimer of different MHC peptide monomers where the heavy chain of the MHC is biotinylated, which allows combination as a tetramer with streptavidine. Such MHC-peptide tetramer has a determined avidity for antibodies that are specific for the epitope peptide and can therefore be used to select the cells that express said antibodies.

In some embodiments, the multimer of the present invention is conjugated with a label. Such labeling allows the skilled person to proceed with immune magnetic enrichment as described herein after. For example, the label is a detectable tag, such as c-Myc, HA, VSV-G, HSV, FLAG, V5, or HIS, which can be detected using an antibody specific to the label, for example, an anti-c-Myc antibody. In some embodiments, the label is a fluorescent molecule. Exemplary fluorescent labels include, but are not limited to, Hydroxycoumarin, Succinimidyl ester, Aminocoumarin, Succinimidyl ester, Methoxycoumarin, Succinimidyl ester, Cascade Blue, Hydrazide, Pacific Blue, Maleimide, Pacific Orange, Lucifer yellow, NBD, NBD-X, R-Phycoerythrin (PE), a PE-Cy5 conjugate (Cychrome, R670, Tri-Color, Quantum Red), a PE-Cy7 conjugate, Red 613, PE-Texas Red, PerCP, Peridinin chlorphyll protein, TruRed (PerCP-Cy5.5 conjugate), Fluor X, Fluoresceinisothyocyanate (FITC), BODIPY-FL, TRITC, X-Rhodamine (XRITC), Lissamine Rhodamine B, Texas Red, Allophycocyanin (APC), an APC-Cy7 conjugate, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, Alexa Fluor 790, Cy2, Cy3, Cy3B, Cy3.5, Cy5, Cy5.5 or Cy7. In some embodiments, the label is allophycocyanin (APC). In some embodiments, the tetramerization is carried out with APC-labeled streptavidins as described in the EXAMPLE.

The cells that express the binding protein with a particular affinity can be screened, sorted and/or enriched according to all methods known to the person skilled in the art. Said enrichment does not kill the labeled cells; therefore cell integrity is maintained for further analysis. In some embodiments, an immunomagnetic enrichment is carried out and typically involves use of magnetic particles (e.g. beads) to allow cell enrichment by contacting the multimers that bind to the cells (i.e. cell that express the binding protein with a determined affinity and/or speficity) with said magnetic particles.

The magnetic particles can be of any shape, including but not limited to spherical, rod, elliptical, cylindrical, disc, and the like. In some embodiments, magnetic particles having a true spherical shape and defined surface chemistry are used to minimize chemical agglutination and non-specific binding. The magnetic particles can be paramagnetic or super-paramagnetic particles. In some embodiments, the magnetic particles are superparamagnetic. Magnetic particles are also referred to as beads herein.

In some embodiments, magnetic particles having a polymer shell are used to protect the target component from exposure to iron. For example, polymer coated magnetic particles can be used to protect target cells from exposure to iron. The magnetic particles can made from well know biocompatible materials. The magnetic particles can range in size from 1 nm to 1 mm. Typically magnetic particles are about 250 nm to about 250 μm in size. In some embodiments, magnetic particle is 0.1 μm to 50 μm in size. In some embodiments, magnetic particle is 0.1 μm to 10 μm in size. In some embodiments, the magnetic particle is a magnetic nano-particle or magnetic microparticle. Magnetic nanoparticles are a class of nanoparticle which can be manipulated using magnetic field. Such particles commonly consist of magnetic elements such as iron, nickel and cobalt and their chemical compounds. Magnetic nano-particles are well known and methods for their preparation have been described in the art, for example in U.S. Pat. Nos. 6,878,445; 5,543,158; 5,578,325; 6,676,729; 6,045,925 and 7,462,446, and U.S. Pat. Pub. Nos.: 2005/0025971; 2005/0200438; 2005/0201941; 2005/0271745; 2006/0228551; 2006/0233712; 2007/01666232 and 2007/0264199, contents of all of which are herein incorporated by reference in their entirety. Magnetic particles are easily and widely available commercially, with or without functional groups capable of binding to affinity molecules. Suitable superparamagnetic particles are commercially available such as from Dynal Inc. of Lake Success, N.Y.; PerSeptive Diagnostics, Inc. of Cambridge, Mass.; Invitrogen Corp. of Carlsbad, Calif.; Cortex Biochem Inc. of San Leandro, Calif.; and Bangs Laboratories of Fishers, Ind. In some embodiments, magnetic particles are Dynal Magnetic beads such as MyOne Dynabeads. In some embodiments, particles are colloidal magnetic particles, e.g. superparamagnetic microparticles having a size of 10 to 200 nm (MACS®; Miltenyi et al, 1990, Cytometry 11:231-238) or micron-sized magnetic particles (e.g. 1-10 μm).

According to the present invention, the surfaces of the magnetic particles are functionalized to attach binding molecules that bind selectively the label conjugated to the multimers as above described. These binding molecules are also referred to as affinity molecules herein. The binding molecule can be bound covalently or non-covalently (e.g. adsorption of molecule onto surface of the particle) to each magnetic particle. Representative examples of binding molecules include, but are not limited to, antibodies, antigens, lectins, proteins, peptides, nucleic acids (DNA, RNA, PNA and nucleic acids that are mixtures thereof or that include nucleotide derivatives or analogs); receptor molecules, such as the insulin receptor; ligands for receptors (e.g., insulin for the insulin receptor); and biological, chemical or other molecules that have affinity for another molecule, such as biotin and avidin. The binding molecules need not comprise an entire naturally occurring molecule but may consist of only a portion, fragment or subunit of a naturally or non-naturally occurring molecule, as for example the Fab fragment of an antibody. The binding molecule may further comprise a marker that can be detected.

In some embodiments, the binding molecule is an aptamer. Aptamers can include, without limitation, defined sequence segments and sequences comprising nucleotides, ribonucleotides, deoxyribonucleotides, nucleotide analogs, modified nucleotides and nucleotides comprising backbone modifications, branchpoints and nonnucleotide residues, groups or bridges. Methods for selecting aptamers for binding to a molecule are widely known in the art and easily accessible to one of ordinary skill in the art.

In some embodiments of the aspects described herein, the binding molecules specific are polyclonal and/or monoclonal antibodies and antigen-binding derivatives or fragments thereof. Well-known antigen binding fragments include, for example, single domain antibodies (dAbs; which consist essentially of single VL or VH antibody domains), Fv fragment, including single chain Fv fragment (scFv), Fab fragment, and F(ab′)2 fragment. Methods for the construction of such antibody molecules are well known in the art. Antibodies or antigen-binding fragments specific for various antigens are available commercially from vendors such as R&D Systems, BD Biosciences, e-Biosciences and Miltenyi, or can be raised against these cell-surface markers by methods known to those skilled in the art.

The binding molecule can be conjugated to the magnetic particle using any of a variety of methods known to those of skill in the art. The affinity molecule can be coupled or conjugated to the magnetic particles covalently or non-covalently. The covalent linkage between the affinity molecule and the magnetic particle can be mediated by a linker. The non-covalent linkage between the affinity molecule and the magnetic particle can be based on ionic interactions, van der Waals interactions, dipole-dipole interactions, hydrogen bonds, electrostatic interactions, and/or shape recognition interactions. In some embodiments, the binding molecule is coupled to the magnetic particle by use of an affinity binding pair. Exemplary binding pairs include any haptenic or antigenic compound in combination with a corresponding antibody or binding portion or fragment thereof (e.g., digoxigenin and anti-digoxigenin; mouse immunoglobulin and goat antimouse immunoglobulin) and nonimmunological binding pairs (e.g., biotin-avidin, biotin-streptavidin, hormone [e.g., thyroxine and cortisol-hormone binding protein, receptor-receptor agonist, receptor-receptor antagonist (e.g., acetylcholine receptor-acetylcholine or an analog thereof), IgG-protein A, lectin-carbohydrate, enzyme-enzyme cofactor, enzyme-enzyme inhibitor, and complementary oligonucleotide pairs capable of forming nucleic acid duplexes), and the like.

Methods to separate cells magnetically are commercially available e.g. from Miltenyi Biotec, Bergisch Gladbach, Invitrogen, Stem cell Technologies, in Cellpro, Seattle, or Advanced Magnetics, Boston. Using MACS® it is possible to enrich from up to 2×10¹⁰ cells in one enrichment step. Other commercially available methods are from Invitrogen, Stem cell Technologies, in Cellpro, Seattle or Advanced Magnetics, Boston. For example, autologous monoclonal antibodies can be directly coupled to magnetic polystyrene particles like Dynal M 450 or similar magnetic particles and used e.g. for cell separation. Alternatively, antibodies can be biotinylated or conjugated with digoxigenin and used in conjunction with avidin or anti-digoxigenin coated affinity columns. In some embodiments, monoclonal antibodies are used in conjunction with colloidal superparamagnetic microparticles having an organic coating by e.g. polysaccharides (Miltenyi et al., 1990). These particles can be used having a size of 10 to 200 nm, preferably between 40 and 100 nm, and can be either directly conjugated to autologous antibodies or used in combination with anti-immunoglobulin, avidin or antihapten-specific microbeads. Polysaccharide-coated superparamagnetic particles are commercially available from Miltenyi Biotec GmbH, Germany.

In some embodiments, the cells are analyzed (characterized) after magnetic pre-enrichment according to all methods known to the person skilled in the art. Preferred for the characterization of cells are in particular, flow cytometry (e.g. fluorescence activated cell sorting (FACS)), ELISA, PCR and/or all fluorescence microscopes known in the art. Particularly preferred is the use of flow-cytometry (FACS). For flow-cytometry, e.g. FACS (fluorescence activated cell sorting), antibodies are used that may be coupled with fluorescence markers that are known to a person of skill in the art, like FITC, phycoerythrin (PE), allophycocyanin (APC), cascade yellow and peridinin chlorophyll protein (PerCP).

In some embodiments, a sequencing step is performed for determining the sequence of the binding domain (e.g. the variable domain of an antibody) that was subjected to mutagenesis. In some embodiments, next generation sequencing is carried out. Some examples of next generation sequencing techniques include, but are not limited to, sequencing by synthesis, sequencing by ligation, and sequencing by hybridization. Examples of next generations sequencing methods include pyrosequencing as used by the GS junior and GS FLX Systems (454 Life Sciences), sequencing by synthesis as used by Illumina's Miseq and Solexa system, the SOLiD™ (Sequencing by Oligonucleotide Ligation and Detection) system (Life Technologies inc.), and ion Torrent Sequencing systems such as the Personal Genome Machine or the Proton Sequencer (Life Technologies Inc), and nanopore sequencing systems (Oxford nanopore). In the case of sequencing by synthesis using Illumina's sequencing technology, the source molecule may be PCR amplified before delivery to a flow cell.

In some embodiments, when the binding protein is an antibody, the sequence encoding for the light or heavy chain is cloned in an appropriate host cell for the production of the binding protein.

In some embodiments, the affinity of the binding protein expressed by a specific population of cells may be determined by according any routine technique well known in the art. Methods for measuring the K_{D} of a binding protein are well known in the art and include, without limitation, surface plasmon resonance (SPR) technology in a BIAcore 3000 instrument. BIACORE® (GE Healthcare, Piscaataway, N.J.) is one of a variety of surface plasmon resonance assay formats that are routinely used to epitope bin panels of monoclonal antibodies. Affinities of antibodies can be readily determined using other conventional techniques, for example, those described by Scatchard et al., (Ann. N.Y. Acad. Sci. USA 51:660 (1949)). For instance binding properties of an antibody to antigens, cells or tissues may generally be determined and assessed using immunodetection methods including, for example, immunofluorescence-based assays, such as immunohistochemistry (IHC) and/or fluorescence-activated cell sorting (FACS). Typically, an antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} for binding to a non-specific antigen (e.g., BSA, casein), which is not identical or closely related to the predetermined antigen. When the K_{D} of the antibody is very low (that is, the antibody has a high affinity), then the K_{D} with which it binds the antigen is typically at least 10,000-fold lower than its K_{D} for a non-specific antigen. An antibody is said to essentially not bind an antigen or epitope if such binding is either not detectable (using, for example, plasmon resonance (SPR) technology in a BIAcore 3000 instrument using a soluble form of the antigen as the ligand and the antibody as the analyte), or is 100 fold, 500 fold, 1000 fold or more than 1000 fold less than the binding detected by that antibody and an antigen or epitope having a different chemical structure or amino acid sequence.

In some embodiments, the cells are subjected to another round of mutagenesis coupled to an affinity-based cell selection and immune magnetic enrichment as disclosed herein wherein the avidity of the multimer for its specific binding protein is decreased between each round. For instance, a first round is carried out with a multimer having a determined number of specific target molecules (e.g. tetramer having 3 specific monomers) and a second round is carried out with a multimer having a decreased number of specific target molecules (e.g. tetramer having only 2 specific monomers). Optionally, a plurality of rounds up to the use of a multimer having only one specific target molecule.

In some embodiments, the concentration of the multimer can also be progressively decreased between two rounds to increase selection stringency.

In this manner, the process lead to a selection of antibodies having an increased affinity and or specificity for its ligand.

### Kits

This invention further provides kits containing reagents for performing the above-described methods, including (i) all component of the gene editing platform as disclosed herein for performing mutagenesis and (ii) all elements for performing the affinity-based selection and immunomagnetic enrichment. To that end, one or more of the reaction components, e.g., RNA molecules, and nucleic acid molecules encoding for the CRISPR/Cas nucleases, the non-nuclease DNA modifying enzyme, for the methods disclosed herein can be supplied in the form of a kit for use. In some embodiments, the kit comprises a nucleic acid encoding for the CRISPR/Cas nuclease, a nucleic acid molecule encoding for the non-nuclease DNA modifying enzyme, and a plurality of RNA molecule described above. In some embodiments, the kit further comprises a plurality of multimers as disclosed herein with different avidity for the binding protein so as to perform the affinity-based selection as well as magnetic particles as disclosed herein for performing the immunomagnetic enrichment.

In some embodiments, the kit can include one or more other reaction components. In some embodiments, an appropriate amount of one or more reaction components is provided in one or more containers or held on a substrate. Examples of additional components of the kits include, but are not limited to, one or more host cells, one or more reagents for introducing foreign nucleotide sequences into host cells, one or more reagents (e.g., probes or PCR primers) for detecting expression of the RNA or protein or verifying the target nucleic acid's status, and buffers or culture media for the reactions. The kit may also include one or more of the following components: supports, terminating, modifying or digestion reagents, osmolytes, and an apparatus for detection.

The reaction components used can be provided in a variety of forms. For example, the components (e.g., enzymes, RNAs, probes and/or primers) can be suspended in an aqueous solution or as a freeze-dried or lyophilized powder, pellet, or bead. In the latter case, the components, when reconstituted, form a complete mixture of components for use in an assay. The kits of the invention can be provided at any suitable temperature. For example, for storage of kits containing protein components or complexes thereof in a liquid, it is preferred that they are provided and maintained below 0° C., preferably at or below −20° C., or otherwise in a frozen state.

A kit or system may contain, in an amount sufficient for at least one assay, any combination of the components described herein. In some applications, one or more reaction components may be provided in pre-measured single use amounts in individual, typically disposable, tubes or equivalent containers. With such an arrangement, a RNA-guided reaction can be performed by adding a target nucleic acid, or a sample or cell containing the target nucleic acid, to the individual tubes directly. The amount of a component supplied in the kit can be any appropriate amount and may depend on the target market to which the product is directed. The container(s) in which the components are supplied can be any conventional container that is capable of holding the supplied form, for instance, microfuge tubes, microtiter plates, ampoules, bottles, or integral testing devices, such as fluidic devices, cartridges, lateral flow, or other similar devices.

The kits can also include packaging materials for holding the container or combination of containers. Typical packaging materials for such kits and systems include solid matrices (e.g., glass, plastic, paper, foil, micro-particles and the like) that hold the reaction components or detection probes in any of a variety of configurations (e.g., in a vial, microtiter plate well, microarray, and the like). The kits may further include instructions recorded in a tangible form for use of the components.

The invention will be further illustrated by the following figures and examples. However, these examples and figures should not be interpreted in any way as limiting the scope of the present invention.

### FIGURES

FIG. 1: Isolation and characterization of human mAb A2Ab. (A) Sorting strategy used to isolate HLA-A2-specific B lymphocytes from donor NO. Cells with the following phenotypic characteristics: CD3−, CD19+ (left panel), both PE and APC labeled HLA-A2 tetramers+(middle panel), HLA-B7 tetramer BV421− (right panel) were isolated and used to produce recombinant antibodies. (B) A2Ab Ab in FIG. 1B and a control anti-pp65-HLA-A*0201 human mAb (Ac-anti pp65-A2) were tested by ELISA against the following peptide-MHC recombinant monomers: pp65-HLA-A*0201 (pp65-A2), Me1A-HLA-A*0201 (Me1A-A2) and pUV-HLA-B*0701 (pUV-B7). C) The specificity of A2Ab was assessed in a Luminex single antigen bead assay. Results are shown in terms of interval MFI. Positivity threshold was set at 1000. (D) The affinity of A2Ab was measured by surface plasmon resonance by flowing various concentrations of pp65-A2 complex over CM5 chip-bound A2Ab.

FIG. 2: Schematic illustration of CRISPR-X. (A) dCas9 associated with a sgRNA containing MS2 hairpins recruits AID* [symbol] fused to MS2 coat protein leading to localized mutations (stars). Mutations can be induced in the sgRNA binding site or upstream or downstream from it, though only downstream mutations are illustrated here. (B) Binding sites for the nine sgRNAs used on the A2Ab heavy chain variable domain coding sequence are shown. Blue and orange colors indicate complementarity to non-coding and coding strands respectively.

FIG. 3: Generation and selection of HEK 293 cells expressing affinity-matured antibodies. (A) Overall strategy for antibody affinity maturation. HEK 293 cells expressing the initial Ab are subjected to CRISPR-X mutagenesis. Cells expressing variant antibodies of higher avidity are enriched using Stringent Tetramer-Associated Magnetic Enrichment (S-TAME) and expanded in vitro (R for “enriched population”, subscript n for round of mutation/selection). Enriched cells are separated by FACS into tetramer positive-staining (R+) and tetramer negative-staining (R−) populations. Multiple rounds of mutation/selection can be performed successively as indicated. (B) Staining of A2Ab-expressing HEK 293 cells with 4A2-tetramers or 3A2/1B7 tetramers as marked after 3 successive transfections for CRISPR-X mutagenesis. Results shown are before the S-TAME step. (C) Staining of cells with tetramer 3A2/1B7 after S-TAME. Results are shown for cells transfected with dCas9, sgRNAs and MS2 AID* [symbol] (R1+ cells, left panel), AID* [symbol] alone (middle panel) and sgRNA alone (right panel). (D) Cells from the R1 population staining positive with the 3A2/1B7 tetramer were isolated by FACS (R1+ cells). Staining of these cells with tetramers 3A2/1B7 (upper left panel) and 1A2/3B7 (upper right panel) is shown. R1+ cells were subjected to a second round of mutagenesis, S-TAME and FACS selection to generate R2+ cells. Staining of R2+ cells with tetramers 3A2/1B7 (lower left panel) and 1A2/3B7 lower right panel) is shown. The number of cells within marked gates is shown between brackets as a percentage of the total cells analysed.

FIG. 4: Web Logo representation of amino acid mutations in the A2Ab heavy chain. WT: starting sequence. R1+, R2+: sequences after one or two rounds of mutation/selection respectively. The height of each letter is proportional to the preference for that amino acid at that site, and letters are colored by amino-acid hydrophobicity. Residue positions are numbered starting from the first amino acid of the leader peptide of the heavy chain. Major mutation sites are indicated by arrows.

FIG. 5: Characterization of evolved antibodies against HLA-A2. (A) ELISA dose-response curves of R2+ mutated mAbs C4.4 and C4.18 compared to A2Ab. (B) The affinity of C4.18 was measured by surface plasmon resonance by flowing various concentrations of pp65-A2 complex over CM5 chip-bound C4.18. (C) Top panel: staining of 721 221 cells which either express HLA-A2 (721 221(A2)) or do not express it (721 221) by A2Ab, C4.4 and C4.18 at 20 μg/mL. MFI are indicated. Lower panel: dose response staining of A2Ab, C4.4 and C4.18 against BLCL HEN expressing HLA-A2. MFI obtained with various concentrations of C4.18 and C4.4 are indicated. (D) The specificity of mutated R2+ mAbs was assessed in a Luminex single antigen bead assay. Results are shown in terms of interval MFI. Positivity threshold was set at 1000.

### EXAMPLE

### Methods

### Donors

Human peripheral blood samples were obtained from anonymous adult donors after informed consent in accordance with the local ethics committee (Etablissement Français du Sang, EFS, Nantes, procedure PLER NTS-2016-08).

### Cell Lines and Culture Conditions

Human embryonic kidney 293A cells were obtained from Thermo Fisher Scientific, San Jose, Calif., USA (R70507). Cells were grown as adherent monolayers in DMEM (4.5g/l glucose) supplemented with 10% FBS, 1% Glutamax (Gibco) and 1% penicillin (10 000 U/ml)/streptomycin (10 000 U/ml) (a mixture from Gibco). The BLCL cell lines HEN (HLA-A*0201/HLA-A*0101), B721 221 and stably transfected HLA-A2 B721 221 (B721 221 A2) were grown in suspension in RPMI medium supplemented with 10% FBS, 1% Glutamax (Gibco) and 1% penicillin (10 000 U/ml)/streptomycin (10 000U/ml) (a mixture from Gibco).

### Plasmid Constructions

Plasmids for mutagenesis were obtained from Addgene: pGH335_MS2-AID*Δ-Hygro (catalogue n° 85406), pX330S-2 to 7 from the Multiplex CRISPR/Cas9 Assembly System kit (n° 1000000055) and pX330A_dCas9-1×7 from the multiplex CRISPR dCas9/Fok-dCas9 Accessory pack (n° 1000000062). The sgRNA scaffolds in the seven latter plasmids were replaced by the sgRNA_2MS2 scaffold from pGH224_sgRNA_2xMS2_Puro (Addgene n° 85413) and guide sequences then introduced into their BbsI sites before Golden Gate assembly. SgRNA design was performed online using Sequence Scan for CRISPR software (http://crispr.dfci.harvard.edu/SSC/). Final plasmids for mutagenesis thus obtained contain expression cassettes for dCas9 and seven sgRNAs. For production of antibodies, VH and VL regions from human antibodies were subcloned respectively in an IgG-Abvec expression vector (FJ475055) and an Iglambda—AbVec expression vector (FJ51647) as previously described[8]. For mammalian display of antibodies as IgG1, VH and VL regions were subcloned into home-made expression vectors derived from the OriP/EBNA1 based episomal vector pCEP4. The VH and VL expression vectors contain a hygromycin B or Zeocin resistance marker respectively, and a transmembrane region encoding sequence exists in the C gamma constant region sequence.

### IgG1 Mammalian Cell Display

Heavy and light chain expression vectors were co-transfected into the 293A cell line at a 1:1 ratio using JetPEI (PolyplusTransfection, Cat. 101-10N) and cultured for 48 h. Selection of doubly transfected cells was performed using Hygromycin B and Zeocin. Antibody surface expression on the selected cells was confirmed by flow cytometry analysis after staining with a PE-labeled goat-anti-human IgG Fc (Jackson ImmunoResearch).

### Peptide MHC Tetramer

The HLA-A*0201—restricted peptides Pp65₄₉₅ (human CMV [HCMV], NLVPMVATV) and MelA27 (melanoma Ag, ELAGIGILTV) and the HLA-B*0702-restricted UV-sensitive peptide (AARGJTLAM; where J is 3-amino-3-(2-nitro)phenyl-propionic acid) were purchased from GL Biochem (Shanghai, China). Soluble peptide MHC monomers used in this study carried a mutation in the α3 domain (A245V), that reduces CD8 binding to MHC class I. Biotinylated HLA-A*0201/MelA₂₇ (HLA-A2/MelA), HLA-A*0201/Pp65₄₉₅ (HLA-A2/Pp65), HLA-B*0702/UV sensitive peptide (HLA-B7/pUV) monomers were tetramerized with allophycocyanin (APC)-labeled premium grade streptavidins (Molecular Probes, Thermo Fischer Scientific, ref S32362) at a molar ratio of 4:1. Where applicable, the avidity of the tetramer for its specific antibody was decreased by mixing specific (ie peptide HLA-A2) and unspecific (ie peptide UV-sensitive HLA-B7) biotinilated monomers before tetramerization with APC-labeled streptavidins at different molar ratios.

### Flow Cytometry Analysis

The specificity and avidity of IgG expressing HEK 293 cells was analysed by flow cytometry. Cells were first stained in PBS containing 0.5% BSA with Ag tetramers for 30 min at room temperature. Anti-PE human IgG was then added at a 1/500 dilution for 15 mn on ice without prior washing. The binding of mutant antibodies was evaluted on 150 000 BLCL cells. Cells were incubated with various concentrations of large-scale purified mAbs diluted in 25 ml of PBS containing 0.5% BSA for 30 min at room temperature. Anti-PE goat anti-human IgG was then added at a 1/500 dilution for 15 min on ice without prior washing.

### Mutagenesis

4×10⁶ anti HLA-A2 IgG-expressing cells were seeded the day before transfection in a 175 cm flask. For each round of mutation, cells were transiently transfected using JET-PRIME (PolyplusTransfection, Cat. 101-10N) with pGH335_MS2-AID*Δ-Hygro together with two other plasmids allowing expression of a total of 9 different sgRNAs along with dCas9 at a ration 1:1:1.

### Affinity-Based Cell Selection and Immunomagnetic Enrichment

After a round of mutagenesis, transfected cells were expanded until confluency over a week. For selection, 10-20×10⁶ cells were washed, resuspended in 0.2 mL of PBS containing 2% BSA and the antigen (i.e. APC HLA-A2 tetramers or mixed APC HLA-A2/HLA-B7 tetramers) and incubated for 30 min at room temperature. The tetramer-stained cells were then positively enriched using APC Ab-coated immunomagnetic beads and columns as previously described[8]. The resulting enriched fraction was stained with an anti human IgG-PE. IgG PE+ and tetramer APC+ were collected on an ARIA cell sorter. The adopted strategy for evolution of mAb A2Ab was as follows: 1) three rounds of mutagenesis; 2) magnetic enrichment with 3A2/1B7 tetramer; 3) FACS sorting of positive cells. Positively selected and sorted mutated HEK 293 underwent two new rounds of mutation using the same sgRNAs before selection with the 1A2/3B7 tetramer.

### Antibody Production

Small and large scale productions were performed as previously described⁸.

### ELISA

HLA-A2/Pp65 monomers were coated O/N at 4° C. in 100 μL of reconstituted ELISA/ELISPOT coating buffer 1× (Affymetrix) at a final concentration of 2 μg/mL in 96-well ELISA plates (Maxisorp, Nunc). Wells were blocked with 10% FBS DMEM medium (Thermo Fischer Scientific) for 2 h at 37° C. Purified mAbs were serially diluted in PBS (starting concentration:100 μg/ml; dilution factor: 3) and incubated for 2 h at RT. An anti-human IgG-HRP Ab (BD Biosciences) was used at 1 μg/mL for detection after incubation for 1 h at RT. The reaction was visualized by the addition of 50 μL chromogenic substrate (TMB, BD biosciences) for 20 min. ODs were read at 450 nm.

### Anti-HLA Antibody Testing (Luminex)

The specificity analysis of the antibody variants was performed using Single Antigen Flow Bead assays according to the manufacturer's protocol (LabScreen single-antigen LS1A04, One Lambda, Inc., Canoga Park, Calif.), exploring 97 class I alleles. The fluorescence of each bead was detected by a Luminex 100 analyser (Luminex, Austin, Tex.), and recorded as the mean fluorescence intensity (MFI). The positivity threshold for the bead MFI was set at 1000 after removal of the background as previously reported[25]. Clinical relevance of pre-transplant donor-specific HLA antibodies was detected by single-antigen flow-beads.

### Surface Plasmon Resonance

Surface Plasmon Resonance (SPR) experiments were performed at 25° C. on a Biacore 3000 apparatus (GE Healthcare Life Sciences, Uppsala, Sweden) on CM5 sensorchips (GE Healthcare). Capture mAbs were immobilized at 10μg/mL by amine coupling using a mixture of N-hydroxysuccinimide and N-ethyl-N′-dimethylaminopropyl carbodiimide, according to the manufacturer's instructions (GE Healthcare), after a 20-fold dilution in 10 mM sodium acetate buffer pH 5. Then, ethanolamine (1M, pH 8.5, GE Healthcare) was injected to deactivate the sensor chip surface. Purified HLA-A*0201 molecules containing the Pp65₄₉₅ peptide were injected at various dilutions over the capture antibodies for 180s at 40 μL/min. A flow cell left blank was used for referencing of the sensorgrams.

### Bioinformatics Analysis

Amplicon preparation: total RNA was purified from 5×10⁶ HEK 293 cells and 1 μg of total RNA was reverse transcribed using Superscript reverse transcriptase (ThermoFisher). cDNA was subsequently amplified using Q5 DNA polymerase and primers targeting VH sequences. Sense and antisense primers include target sequences suitable for nextera indexage. Barcodes were further introduced by PCR with indexed nextera and the amplicons were sequenced at the IRIC's Genomics Core Facility at Montreal. Paired-end MiSeq technology (Miseq Reagent Nano kit v2 (500 cycles) from Illumina, Inc. San Diego, Calif., USA) was used, with a 2×250 bp setup.

### Pretreatment and Sequence Clustering

For each chip generated, approximately one million reads were obtained for all the samples. The quality and length distribution of the reads were checked using the FASTQ tool (v0.11.7). After that, for each sample, the paired-end sequences were assembled using the PEAR software (v0.9.6) while keeping only the sequences whose Phred score was greater than 33 and whose overlap was at least 10 nucleotides. Then 30000 sequences were randomly selected to normalize samples. Next, for each sample, full length VH sequences were grouped according to their identity and counted and clusters were formed as described in the text. Mutations observed in the mock control (gRNA only) experiment were then eliminated in order to distinguish site-directed mutations from RT-PCR or sequence errors. Only clusters representing more than 0.1% of the total number of sequences were retained.

### Alignment and Mutation Analysis

For each sample, the generated clusters were annotated by aligning each sequence cluster against the reference sequence using Biostring library (v2.48.0) in a custom R script, to generate a counting table. The generated data were filtered by subtracting the mutations detected in the mock sample. A position matrix was then generated to create a Weblogo using the ggseqlogo library (v0.1). All statistical analyses were performed in a custom R script.

### Results

### Isolation of a Low Affinity Human Antibody Against HLA-A*0201

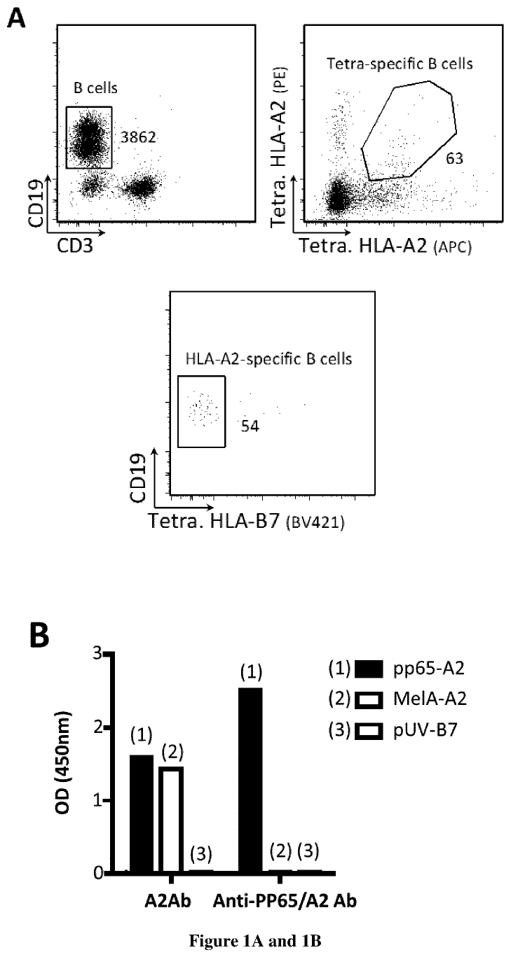

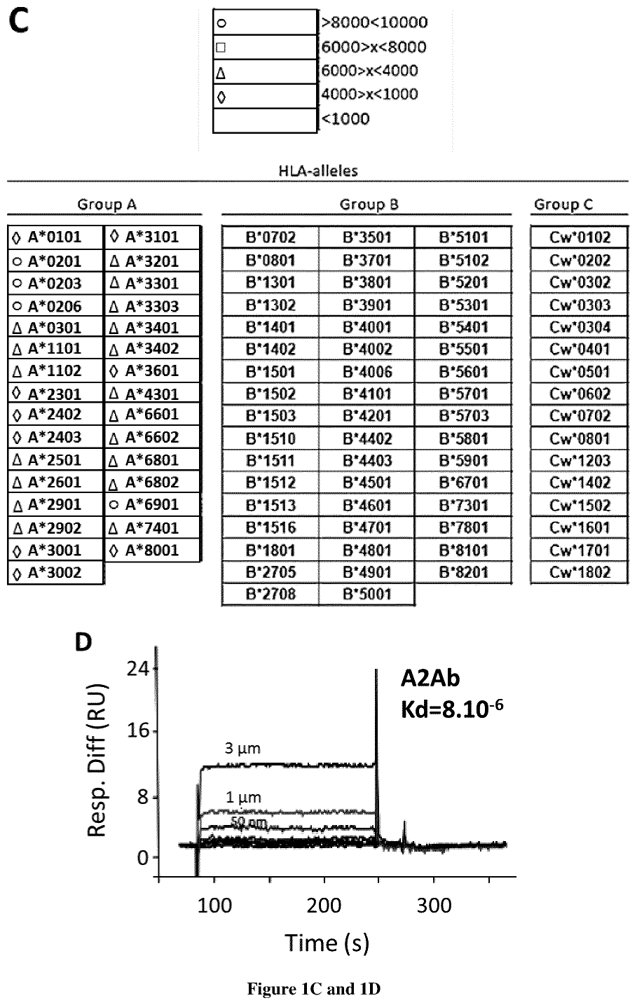

A human HLA-A*0201 molecule (hereafter referred to as HLA-A2) was selected as a target for antibody discovery and maturation as it is easy to obtain blood samples from donors not previously immunized against this MHC allele. In addition, various recombinant HLA molecules were readily available in our laboratory. PBMCs from three HLA-A2-negative donors with negative serology for HLA-A2 circulating antibodies were tested for the presence of blood circulating B cells specific for HLA-A2. This was done by flow cytometry sorting of B cells that bound HLA-A2 tetramers labeled with two different fluorochromes but did not bind HLA-B7 tetramers, using a technique described previously[8, 10]. B lymphocytes stained specifically by HLA-A2 tetramers could be identified in PBMC from all three donors (see FIG. 1A for an example) and were isolated as single cells. We attempted RT-PCR amplification of sequences coding for the variable regions of the heavy and light chains of four B lymphocytes isolated from one donor (NO) using a recently published protocol[8, 10]. A pair of heavy and light chain V region coding sequences was obtained for one of the four cells. After cloning these gene segments into eukaryotic expression vectors in phase with human heavy and light chain constant domains, the corresponding antibody (A2Ab) was successfully produced in the supernatant of transfected HEK cells and tested for its specificity. A2Ab recognizes HLA-A2 but not HLA-B7 in ELISA tests and this recognition does not depend on the peptide loaded into the HLA pocket (FIG. 1B). A single HLA antigen flow bead assay analysis confirmed that A2Ab can recognize HLA-A*0201, but also showed that A2Ab recognizes closely related alleles belonging to the HLA-A*02 supertype (HLA-A*0203, A*0206 and A*6901) and weakly cross-reacts with other MHC A alleles. However, B or C alleles are not recognized (data not shown, results summarized in FIG. 1C). Finally, the affinity of A2Ab for the pp65/HLA-A2 complex was determined by surface plasmon resonance (SPR) to be in the low micromolar range (Kd=8.10⁻⁶, FIG. 1D). This is consistent with the HLA-A2-specific B cells being isolated from a naive/non-immune blood circulating B cell repertoire.

### CRISPR-X Targeted Mutagenesis of A2Ab and Screening for Higher Avidity Antibodies

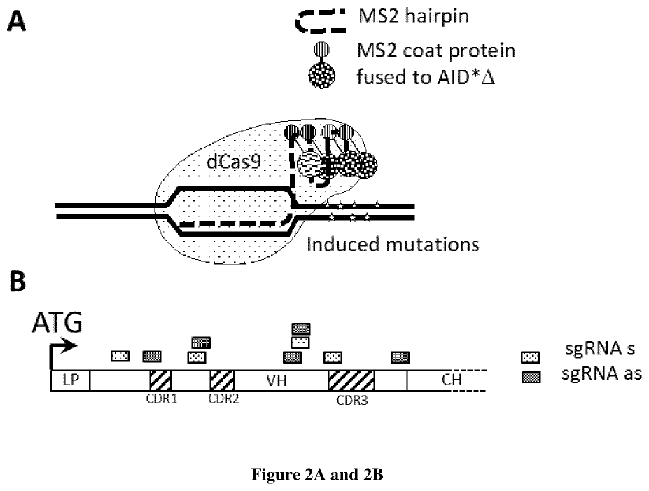

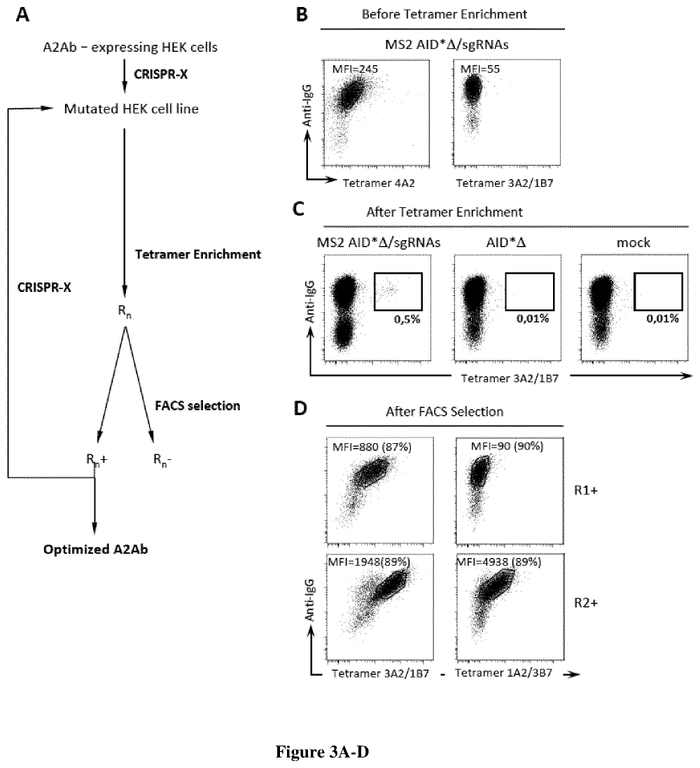

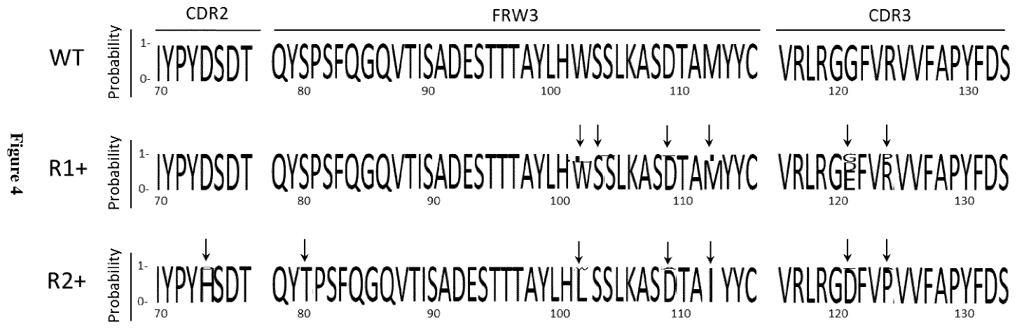

We used the CRISPR-X approach[24] (FIG. 2A) to mutate the A2Ab sequence. Our overall procedure using iterative mutation and selection is summarized in FIG. 3A. HEK 293 cells were engineered to express cell surface A2Ab by stable transfection of episomal vectors expressing its heavy and light chains (HC and LC, respectively). For induction of mutations, these cells were then transiently transfected with a plasmid coding for AID*Δ fused to MS2 coat protein, and plasmids coding for dCas9 and nine different sgRNAs spanning the sequence coding for the A2Ab HC variable domain (FIG. 2B). AID*Δ is an AID mutant with increased SHM activity whose Nuclear Export Signal (NES) has been removed[24]. It has significantly increased mutation activity compared to wild-type AID without a NES[24]. Three successive transient transfections were performed before cells were screened for expression of mutant antibodies with increased avidity for HLA-A2.

Cells we started from stably expressed cell surface A2Ab and thus were able to bind tetramers comprising four HLA-A2 molecules. These cells were subjected to three successive transfections. We expected cells expressing higher avidity antibodies post-mutagenesis to be able to bind tetramers containing fewer HLA-A2 molecules. We thus sought to identify cells in the mutated polyclonal population using labeling with a tetramer made up of 3 HLA-A2 molecules and one B7 molecule (3A2/1B7). As shown in FIG. 3B, we were unable to detect any 3A2/1B7-labeled cells in the mutated polyclonal population by flow cytometry, while all cells expressing IgG were labeled with the initial tetramer (4A2) as expected.

We suspected that 3A2/1B7-labeled cells might be too rare to be detectable in the fraction of the mutated polyclonal population we tested, so we tried to enrich them before analysis. The mutated poylconal population was first incubated with the 3A2/1B7 tetramer coupled to APC, then subjected to positive selection using paramagnetic beads coupled to anti-APC antibodies. After magnetic enrichment, we observed a small proportion of cells clearly labeled by the 3A2/1B7 tetramer (FIG. 3C, left dot-plot). Notably, no such cells were detected when our protocol was carried out using A2Ab-expressing HEK 293 cells transfected with a hyperactive non-guided AID (FIG. 3C, middle dot-plot), or with guide RNAs alone (“mock”, FIG. 3C, right dot-plot). This first “positive” population (R1) was purified by cell sorting and expanded in vitro to yield population R1+ (>95% pure). In marked contrast to the starting population, the R1+ population bound tetramers with just 3 HLA-A2 molecules (3A2/1B7, FIG. 3D, upper left dot-plot).

To complete a further round of mutagenesis/selection, we exposed the R1+ population to two successive transfections for mutagenesis using the same batch of sgRNAs as above, before selection was performed. This time we used a more stringent enrichment process with tetramers containing only one HLA-A2 molecule (1A2/3B7). A new population of tetramer positive cells was obtained (R2+), with a 2.2 fold increase in the 3A2/1B7 tetramer mean fluorescence intensity compared to R1+ (FIG. 3D, bottom left dot-plot). The R2+ population was also stained by tetramer 1A2/3B7, in marked contrast to R1+ cells (FIG. 3D, compare upper and lower right dot-plots). Each round of mutation and selection thus increases the avidity of the antibodies.

### Antibody Sequence Evolution During Mutagenesis and selection Rounds

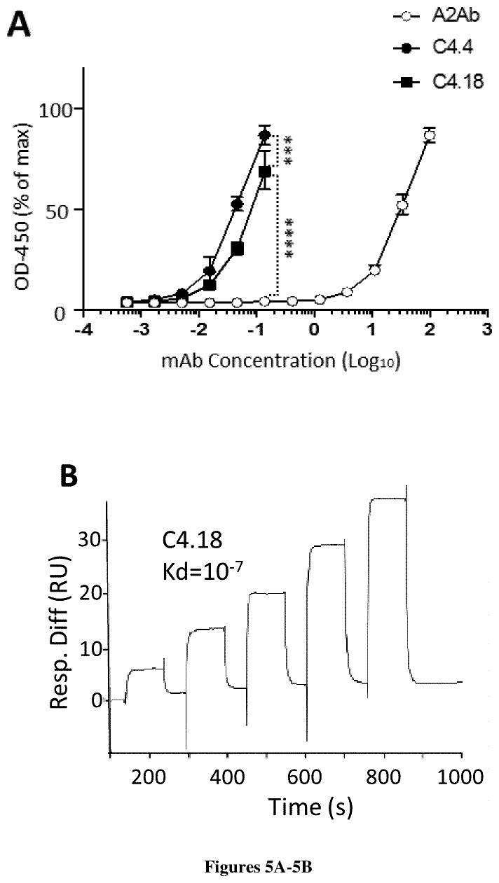

As described above, we were unable to detect cells capable of binding to the 3A2/1B7 tetramer after one round of mutagenesis until we used magnetic enrichment. This enrichment generated the R1 population. FACS sorting of this population yielded the R1+ population capable of binding 3A2/1B7 tetramers and the R1− population incapable of binding this tetramer. We used next generation sequencing (NGS) to search for heavy chain sequences enriched in the R1+ population relative to the R1− population and which could contain mutations responsible for the increased affinity of the R1+ population antibodies. 30,000 randomly selected reads from each population were analyzed. Reads represented more than 50 times were placed into a read-specific cluster, while reads represented less than 50 times were grouped together in a category we termed “small clusters”. For the R1+ population, two large clusters representing together 42.5% of reads were detected, in addition to a third large cluster representing WT sequences (Table I). Six other clusters representing together 5.2% of reads were also detected, together with numerous reads in the small cluster category. Seven of these eight non-WT clusters were clearly under-represented in the R1− population, where the WT cluster and small clusters predominated. Mutations observed in the seven clusters were located in the FR3 and CDR3 regions (FIG. 4). They were often shared between different clusters, suggesting that they contribute to the increased affinity of R1+ population antibodies.

That WT and small cluster sequences represent 52.6% of R1+ reads might seem surprising. However, in the HEK 293 cells subjected to mutagenesis, antibody genes are present on episomal vectors, with several vector copies per cell[26]. Cells selected with the 3A2/1B7 tetramer may contain only one gene copy with a mutation leading to an antibody of increased affinity. All the other copies could contain either no mutation or neutral or even deleterious mutations, yet they will be co-enriched with the copy carrying the affinity-increasing mutation.

The second round of mutation/selection led to a drastic decline in WT reads (from 13.6% for R1+, to 0% for R2+), while in the R2+ population a cluster representing nearly half of the NGS reads emerged, corresponding to HCs accumulating six mutated amino acids: D74H/S80T/W102L/M112I/G121D/R124P (Table II, FIG. 4). Interestingly, the CDR2 D74H mutation was not detected in the R1+ population. Nine of the thirteen R2+ clusters (a cluster contains more than 50 reads of the cluster-specific sequence) differ only very slightly from this main sequence, underlining a strong convergence of most of the R2+ clusters. The W102, M112I, G121D and R124P mutations were already well represented in the R1+ population (Table I). The second round of mutation/selection led to emergence of two new R2+-specific mutations: D74H in the CDR2 and S8OT in the FR3 region.

### Characterization of Evolved Antibodies against HLA-A2

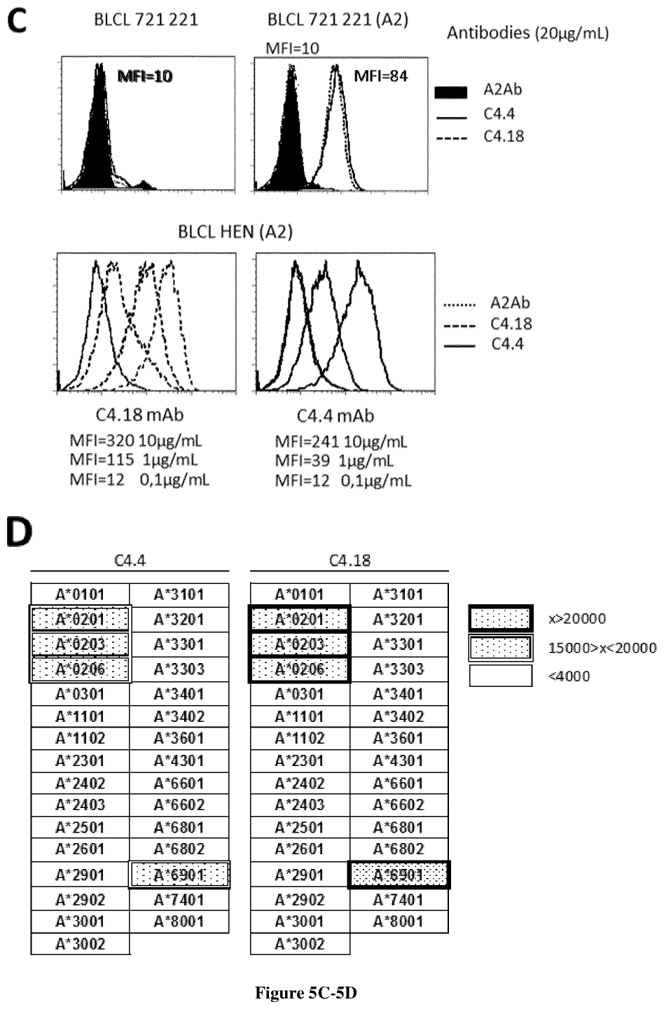

The R2+ antibodies C4.4 and C4.18 (Tables I and II) were produced as recombinant proteins for comparison of their affinity and specificity to those of the initial A2Ab. As shown in FIG. 5A, C4.4 and C4.18 mAbs show clearly increased reactivity against HLA-A*0201 compared to A2Ab in an ELISA. We next determined C4.18's affinity for HLA-A*0201 by SPR: Kd=10⁻⁷ (FIG. 5B). This is an almost two log increase over that of the initial A2Ab (Kd=8×10⁻⁶). We were unable to make enough C4.4 for SPR studies.

These results demonstrate that our matured antibodies bind with higher affinity to antigen than A2Ab in fully in vitro tests. But can they bind to antigen expressed on the surface of cells, a prerequisite for biological activity? The initial A2Ab was not of sufficient affinity to bind to two HLA-A2 expressing cell lines tested, 721.221 B cells made HLA-A2 positive by transfection (721.221(A2)), and naturally HLA-A2 expressing BLCL HEN. However, the increased affinity of C4.4 and C4.18 led to ready detection of such binding (FIG. 5C). Binding to 721.221(A2) B cells was HLA-A2 dependent, as no binding was observed to the parental HLA-A2 negative 721.211 B cells. A single HLA antigen flow bead assay analysis confirmed that C4.4 and C4.18 had higher affinity than A2Ab for HLA-A*0201 and also showed a gain in specificity, as they had significantly less crossreactivity against other HLA-A alleles (compare FIG. 5D to FIG. 1C).

### Discussion

We show that starting from a low affinity antibody, CRISPR-X targeting of AID to antibody genes can be used to obtain affinity-matured human antibodies in cellulo in about 6 weeks. Thus we increased the affinity of a fully human anti-HLA-A*0201 mAb to sufficient levels for biological activity and without loss of specificity in just 2 cycles of mutation/selection (each cycle consisting of several successive mutagenesis transfections prior to the selection steps). The low affinity antibody we started from was expressed by naive B cells. Our procedure thus mimics in vitro antibody maturation in secondary lymphoid organs, where naive B lymphocytes stimulated by Ag recognition via specific BCRs of limited affinity go on to generate receptors optimized for Ag recognition.

Using SHM for in vitro affinity maturation of antibodies is an attractive strategy and has been used previously in a variety of cell lines [2, 27-30]. Some recently described technologies to affinity-mature antibodies in vitro rely on the integration of a library of CDR3 domains using CRISPR Cas9 technology[31] or mutagenesis of only the most permissive CDR positions[32]. Prior to these approaches, the Bowers group pioneered the coupling of AID-induced somatic hypermutation with mammalian cell surface display in the easily transfectable HEK 293 cells for in vitro maturation of mAbs[15]. We have extended this latter approach to include specific targeting of AID to the immunoglobulin genes to be mutated using a combination of dCas9-AID fusions and specific guide RNAs. We have also introduced a magnetic enrichment step prior to FACS sorting of mutated cells to facilitate isolation of cells expressing higher affinity antibodies. These modifications proved necessary to obtain our affinity matured anti-HLA antibodies after only 2 rounds of mutation/selection. Indeed, we were unable to detect any cells carrying higher affinity antibodies when AID activity was not targeted to the Ig sequences, and we could only detect and isolate them after the first mutation round if magnetic enrichment preceded FACS sorting.

While this manuscript was in preparation, Liu et al. described a variety of diversifying base editors and showed that they retained their intrinsic nucleotide preferences when recruited to DNA as MS2 coat fusions. They also demonstrated that it was possible to use diversifying base editors to affinity mature a previously studied murine anti-4-hydroxy-3-nitrophenylacetyl (NP) antibody called B1-8³¹. The matured antibodies they obtained contained various mutations that had already been observed after subjecting B1-8 to SHM in a mouse in vivo immunization model. The effect of these point mutations was tested separately, and it was not clear whether any of their antibodies contained multiple mutations. In our study, we define previously unknown combination of mutations that are required to increase the affinity of a human antibody against HLA-A2, without loss of specificity. As might be expected, “beneficial” mutations could be found in the CDR2 and CDR3. Interestingly, CDR3 mutations appeared after the first round of mutation/selection, while CDR2 mutations only appeared after the second round. In addition to the CDR2 and CDR3 mutations, some mutations also appeared in the FR3. In particular, the C4.18 mAb obtained after the second round of mutagenesis differs from the first round C3.9 mAb by only two additional mutated amino acids located in FR3. This is interesting as antibody in vitro evolution studies have suggested that mutations leading to higher affinity often correspond to residues distant from the antigen binding site and that affinity maturation of antibodies occurs most effectively by changes in second sphere residues rather than contact residues[33, 34]. It is also interesting to note that increasing the affinity of our antibodies for HLA-A*0201 also led to an increase in their specificity: they progressively lost their crossreactivity against non-HLA-A*02 alleles.

The progressive evolution of A2Ab we observed, with a gradual accumulation of combinations of mutations, is probably necessary for the maturation of the affinity of most antibodies. The combination of CDR and FR mutations could result from CRISPR-X allowing simultaneous targeting of multiple sites all along the Ig variable sequence and potentially represents an important advantage over other recently described technologies limiting mutagenesis to the CDR3[31] or to the most permissive CDR positions[32].

Our CRISPR-X based approach can readily be developed further to increase the potential for antibody diversification. We used the same 9 gRNAs for both rounds of mutagenesis. Further rounds of mutagenesis could be carried out using different gRNAs. The CRISPR-X approach using S. pyogenes dCas9 requires the presence of an NGG PAM immediately downstream from the gRNA binding site. Cas9 variants with relaxed PAM requirements could also be used in this approach, including the recently described variant using a PAM reduced to NG. This would lift almost all constraints on gRNA choice. We focused on mutating the Ig heavy chain gene alone, but both heavy and light chain genes were present in cells subjected to mutagenesis. We did not detect any light chain mutations after transfection of the heavy chain gRNAs (data not shown), demonstrating the specificity of the targeting approach. However, AID could be targeted simultaneously to both heavy and light chain genes by cotransfecting cells with a mixture of heavy and light chain gRNAs, increasing the diversification possibilities by association of mutated heavy and light chains in different combinations.

The A2Ab mAb used here served as an initial proof of concept for antibody maturation in vitro using CRISPR-X. However, the fully human mAbs specific for the HLA-A*0201 allele we generated could have direct clinical applications, notably in the context of mismatch HLA-A2 organ transplantation. Two recent studies described the efficacy of anti-HLA-A2-specific CARs of murine origin in the control of graft rejection in animal models[35, 36]. Using fully human antibodies could be an important step forward for implementation of such strategies to humans. Furthermore, the availability of a series of mAbs of increasing affinity (derived from different rounds of mutation/selection) could be useful to study the impact of CAR affinity on biological activity and could also help to improve predictive algorythms for antibody maturation.

In conclusion, we describe a new approach for progressive and controlled antibody evolution. This procedure should allow us to obtain antibodies of high affinity and specificity against virtually any Ag, if available in a recombinant form, starting directly from circulating naïve B cells, which represent a vast pool of Ag-specific antibodies to tap into. Our approach may prove particularly useful when fully human antibodies are required: when first isolated from non-immunized individuals, they are often of insufficient affinity for therapeutic or research purposes. Many Ag of interest for the treatment of pathologies such as cancer are in this category and thus represent potential targets for this approach. In addition, our approach can be adapted to optimize antibody specificity by addition of a simple negative selection step to eliminate antibodies with undesired interactions. This could be useful for improving the specificity of currently existing murine, chimeric or humanized antibodies.

### TABLES

TABLE I
CRISPR-X-mediated evolution of A2Ab: NGS analysis, round 1
R1

| | mAb | % R1+ | | % R1− | |
|---|---|---|---|---|---|
| Cluster name | name | (counts) | | (counts) | |
| G121E | C3.2 | 31.8 | (9542) | 0.3 | (94) |
| WT | A2Ab | 13.6 | (4103) | 52.6 | (15788) |
| W102L//M112I//G121D//R124P | C3.9 | 10.7 | (3197) | 0 | |
| G121E//V140L | | 1.1 | (340) | 0 | |
| G121D | C3.3 | 1.1 | (316) | 0.3 | (95) |
| S103N//G121D | C3.5 | 0.9 | ( 261) | 0 | |
| W1021//D109A//M112I// | | 0.8 | (239) | 0 | |
| G121D//R124P | | | | | |
| M112I//G121D//R124P | | 0.7 | (209) | 0 | |
| V140L | | 0.6 | (168) | 1.5 | (448) |
| R117S | | 0 | | 0.5 | (148) |
| Y114S | | 0 | | 0.4 | (124) |
| D109A | | 0 | | 0.4 | (119) |
| S103R | | 0 | | 0.2 | (67) |
| S108A | | 0 | | 0.2 | (66) |
| G137R | | 0 | | 0.2 | (61) |
| R119S | | 0 | | 0.2 | (60) |
| P60A | | 0 | | 0.2 | (54) |
| V123G | | 0 | | 0.2 | (54) |
| small clusters R1+ (number) | C3.4 | 38.7 | (11625) | | |
| small clusters R1− (number) | | | | 42.7 | (12817) |
| total | | 100 | (30000) | 100 | (30000) |

TABLE II
CRISPR-X-mediated evolution of A2Ab: NGS analysis, round 2
R2

| | mAb | % R2+ | | % R2− | |
|---|---|---|---|---|---|
| Cluster name | name | (counts) | | (counts) | |
| D74H//S8OT//W102L//M112I// | C4.4 | 49.2 | (14755) | 9.2 | (2756) |
| G121D//R124P | | | | | |
| D74H//S8OT//W102L//D109A// | | 2.4 | (733) | 0.2 | (73) |
| M112I//G121D//R124P | | | | | |
| D74H//S8OT//M112I// | | 2.2 | (650) | 0 | |
| G121D//R124P | | | | | |
| G121E | | 1.7 | (496) | 38.9 | (11670) |
| D74H//S8OT//F83S//W102L// | | 0.7 | (223) | 0.4 | (112) |
| M112I//G121D//R124P | | | | | |
| D74H//S8OT//A98P//W102L// | | 0.6 | (182) | 0 | |
| M112I//G121D//R124P | | | | | |
| D74H//S8OT//W102L//M112I// | | 0.6 | (173) | 0 | |
| G121D//R124P//V140L | | | | | |
| D74H//W102L//M112I// | C4.18 | 0.5 | (163) | 0 | |
| G121D//R124P | | | | | |
| G121D//R124P | | 0.2 | (74) | 0.9 | (274) |
| D74H//S8OT//W102L//S104T// | | 0.2 | (72) | 0 | |
| M112I//G121D//R124P | | | | | |
| D74H//S8OT//W102L//G121E | | 0.2 | (68) | 0 | |
| W102L//M112I//G121D//R124P | | 0.2 | (63) | 4.2 | (1247) |
| D74H//S8OT//W102L//L105R// | | 0.2 | (59) | 0 | |
| M112I//G121D//R124P | | | | | |
| W52C//G121E | | 0 | | 1.1 | (333) |
| G121E//V140L | | 0 | | 0.7 | (209) |
| R47S//R57H//G121E | | 0 | | 0.7 | (204) |
| W102L//G121E | | 0 | | 0.7 | (203) |
| WT | A2Ab | 0 | | 0.6 | (193) |
| M112I//G121D//R124P | | 0 | | 0.5 | (137) |
| W102L//M112I//G121E | | 0 | | 0.4 | (128) |
| H101Q//G121E | | 0 | | 0.4 | (122) |
| I39M//H101Q//G121E | | 0 | | 0.4 | (119) |
| P60S//G121E | | 0 | | 0.4 | (81) |
| C41Y//G121E | | 0 | | 0.2 | (66) |
| I39M//G121E | | 0 | | 0.2 | (56) |
| W102C//G121E | | 0 | | 0.2 | (56) |
| small clusters R+ (number) | | 41 | (12289) | | |
| small clusters R2− (number) | | | | 39.9 | (11961) |
| total | | 100 | (30000) | 100 | (30000) |

### REFERENCES

Throughout this application, various references describe the state of the art to which this invention pertains. The disclosures of these references are hereby incorporated by reference into the present disclosure.
1. Schatz D G, Ji Y: Recombination centres and the orchestration of V(D)J recombination. Nat Rev Immunol 2011, 11(4):251-263.
2. Martin A, Scharff M D: Somatic hypermutation of the AID transgene in B and non-B cells. Proceedings of the National Academy of Sciences of the United States of America 2002, 99(19):12304-12308.
3. Muramatsu M, Kinoshita K, Fagarasan S, Yamada S, Shinkai Y, Honjo T: Class switch recombination and hypermutation require activation-induced cytidine deaminase (AID), a potential RNA editing enzyme. Cell 2000, 102(5):553-563.
4. Williams S C, Frippiat J P, Tomlinson I M, Ignatovich O, Lefranc M P, Winter G: Sequence and evolution of the human germline V lambda repertoire. J Mol Biol 1996, 264(2):220-232.
5. Calis J J, Rosenberg B R: Characterizing immune repertoires by high throughput sequencing: strategies and applications. Trends Immunol 2014, 35(12):581-590.
6. Corti D, Langedijk J P, Hinz A, Seaman M S, Vanzetta F, Fernandez-Rodriguez B M, Silacci C, Pinna D, Jarrossay D, Balla-Jhagjhoorsingh S et al: Analysis of memory B cell responses and isolation of novel monoclonal antibodies with neutralizing breadth from HIV-1-infected individuals. PloS one 2010, 5(1):e8805.
7. Corti D, Voss J, Gamblin S J, Codoni G, Macagno A, Jarrossay D, Vachieri S G, Pinna D, Minola A, Vanzetta F et al: A neutralizing antibody selected from plasma cells that binds to group 1 and group 2 influenza A hemagglutinins. Science 2011, 333(6044):850-856.
8. Devilder M C, Moyon M, Saulquin X, Gautreau-Rolland L: Generation of Discriminative Human Monoclonal Antibodies from Rare Antigen-specific B Cells Circulating in Blood. J Vis Exp 2018(132).
9. Franz B, May K F, Jr., Dranoff G, Wucherpfennig K: Ex vivo characterization and isolation of rare memory B cells with antigen tetramers. Blood 2011, 118(2):348-357.
10. Ouisse L H, Gautreau-Rolland L, Devilder M C, Osborn M, Moyon M, Visentin J, Halary F, Bruggemann M, Buelow R, Anegon I et al: Antigen-specific single B cell sorting and expression-cloning from immunoglobulin humanized rats: a rapid and versatile method for the generation of high affinity and discriminative human monoclonal antibodies. BMC Biotechnol 2017, 17(1):3.
11. Hoogenboom H R: Selecting and screening recombinant antibody libraries. Nature biotechnology 2005, 23(9):1105-1116.
12. Akamatsu Y, Pakabunto K, Xu Z, Zhang Y, Tsurushita N: Whole IgG surface display on mammalian cells: Application to isolation of neutralizing chicken monoclonal anti-IL-12 antibodies. Journal of immunological methods 2007, 327(1-2):40-52.
13. Al-Qaisi T S, Su Y C, Roffler S R: Transient AID expression for in situ mutagenesis with improved cellular fitness. Sci Rep 2018, 8(1):9413.
14. An L, Chen C, Luo R, Zhao Y, Hang H: Activation-Induced Cytidine Deaminase Aided In Vitro Antibody Evolution. Methods in molecular biology 2018, 1707:1-14.
15. Bowers P M, Horlick R A, Neben T Y, Toobian R M, Tomlinson G L, Dalton J L, Jones H A, Chen A, Altobell L, 3rd, Zhang X et al: Coupling mammalian cell surface display with somatic hypermutation for the discovery and maturation of human antibodies. Proceedings of the National Academy of Sciences of the United States of America 2011, 108(51):20455-20460.
16. Ho M, Nagata S, Pastan I: Isolation of anti-CD22 Fv with high affinity by Fv display on human cells. Proceedings of the National Academy of Sciences of the United States of America 2006, 103(25):9637-9642.
17. Ho M, Pastan I: Display and selection of scFv antibodies on HEK-293T cells. Methods in molecular biology 2009, 562:99-113.
18. McConnell A D, Do M, Neben T Y, Spasojevic V, MacLaren J, Chen A P, Altobell L, 3rd, Macomber J L, Berkebile A D, Horlick R A et al: High affinity humanized antibodies without making hybridomas; immunization paired with mammalian cell display and in vitro somatic hypermutation. PloS one 2012, 7(11):e49458.

19. Su Y C, Al-Qaisi T S, Tung H Y, Cheng T L, Chuang K H, Chen B M, Roffler S R: Mimicking the germinal center reaction in hybridoma cells to isolate temperature-selective anti-PEG antibodies. mAbs 2014, 6(4):1069-1083.
20. Wang L, Jackson W C, Steinbach P A, Tsien R Y: Evolution of new nonantibody proteins via iterative somatic hypermutation. Proceedings of the National Academy of Sciences of the United States of America 2004, 101(48):16745-16749.
21. Hwang J K, Alt F W, Yeap L S: Related Mechanisms of Antibody Somatic Hypermutation and Class Switch Recombination. Microbiol Spectr 2015, 3(1):MDNA3-0037-2014.
22. Hess G T, Tycko J, Yao D, Bassik M C: Methods and Applications of CRISPR-Mediated Base Editing in Eukaryotic Genomes. Mol Cell 2017, 68(1):26-43.
23. Rees H A, Liu D R: Base editing: precision chemistry on the genome and transcriptome of living cells. Nature reviews Genetics 2018.
24. Hess G T, Fresard L, Han K, Lee C H, Li A, Cimprich K A, Montgomery S B, Bassik M C: Directed evolution using dCas9-targeted somatic hypermutation in mammalian cells. Nat Methods 2016, 13(12):1036-1042.
25. Amico P, Honger G, Mayr M, Steiger J, Hopfer H, Schaub S: Clinical relevance of pretransplant donor-specific HLA antibodies detected by single-antigen flow-beads. Transplantation 2009, 87 (11): 1681-1688.
26. Yates J L, Warren N, Sugden B: Stable replication of plasmids derived from Epstein-Barr virus in various mammalian cells. Nature 1985, 313(6005):812-815.
27. Cumbers S J, Williams G T, Davies S L, Grenfell R L, Takeda S, Batista F D, Sale J E, Neuberger M S: Generation and iterative affinity maturation of antibodies in vitro using hypermutating B-cell lines. Nature biotechnology 2002, 20(11):1129-1134.
28. Delker R K, Fugmann S D, Papavasiliou F N: A coming-of-age story: activation-induced cytidine deaminase turns 10. Nat Immunol 2009, 10(11):1147-1153.

29. Maul R W, Gearhart P J: AID and somatic hypermutation. Adv Immunol 2010, 105:159-191.
30. Seo H, Hashimoto S, Tsuchiya K, Lin W, Shibata T, Ohta K: An ex vivo method for rapid generation of monoclonal antibodies (ADLib system). Nat Protoc 2006, 1(3):1502-1506.
31. Mason D M, Weber C R, Parola C, Meng S M, Greiff V, Kelton W J, Reddy S T: High-throughput antibody engineering in mammalian cells by CRISPR/Cas9-mediated homology-directed mutagenesis. Nucleic Acids Res 2018, 46(14):7436-7449.
32. Tiller K E, Chowdhury R, Li T, Ludwig S D, Sen S, Maranas C D, Tessier P M: Facile Affinity Maturation of Antibody Variable Domains Using Natural Diversity Mutagenesis. Front Immunol 2017, 8:986.
33. Boder E T, Midelfort K S, Wittrup K D: Directed evolution of antibody fragments with monovalent femtomolar antigen-binding affinity. Proceedings of the National Academy of Sciences of the United States of America 2000, 97(20):10701-10705.
34. Persson H, Kirik U, Thornqvist L, Greiff L, Levander F, Ohlin M: In Vitro Evolution of Antibodies Inspired by In Vivo Evolution. Front Immunol 2018, 9:1391.
35. MacDonald K G, Hoeppli R E, Huang Q, Gillies J, Luciani D S, Orban P C, Broady R, Levings M K: Alloantigen-specific regulatory T cells generated with a chimeric antigen receptor. J Clin Invest 2016, 126(4):1413-1424.
36. Noyan F, Zimmermann K, Hardtke-Wolenski M, Knoefel A, Schulde E, Geffers R, Hust M, Huehn J, Galla M, Morgan M et al: Prevention of Allograft Rejection by Use of Regulatory T Cells With an MHC-Specific Chimeric Antigen Receptor. Am J Transplant 2017, 17(4):917-930.

1. A method of generating and selecting a variant of a binding protein with increased binding affinity and/or specificity for a binding domain comprising
subjecting a population of cells that express the binding protein to at least one round of mutagenesis coupled to an affinity/specificity-based cell selection and immunomagnetic enrichment.

2. The method of claim 1 wherein the population of cells express an antibody at the cell surface.

3. The method of claim 1, wherein the population of cells is a population of B cells or a population of eukaryotic cells engineered for expressing an antibody.

4. The method of claim 3 wherein the antibody that is expressed by the population of cells is a whole antibody having 2 light chains and 2 heavy chains.

5. The method of claim 1 wherein the mutagenesis comprises contacting the population of cells that express the binding protein with a gene editing platform (a) a defective CRISPR/Cas nuclease engineered for sequence targeting, (b) a non-nuclease DNA modifying enzyme and (c) a plurality of RNA molecules for guiding the defective CRISPR/Cas nuclease and the non-nuclease DNA modifying enzyme to a plurality of target sequences in a DNA nucleic acid molecule coding for the binding domain of the binding protein.

6. The method of claim 5 wherein the defective CRISPR/Cas nuclease is a mutant Cas9 protein from S. pyogenes.

7. The method of claim 5 wherein the defective CRISPR/Cas nuclease comprises the amino acid sequence as set forth in SEQ ID NO: 1.

8. The method of claim 5 wherein the non-nuclease DNA modifying enzyme has the activity of cytosine deaminases, adenosine deaminases, DNA methyltransferases, and/or DNA demethylases.

9. The method of claim 8 wherein the non-nuclease DNA modifying enzyme derives from Activation Induced cytidine Deaminase (AID).

10. The method of claim 8 wherein the non-nuclease DNA modifying enzyme is the AID*Δ that has the amino acid sequence as set forth in SEQ ID NO:2.

11. The method of claim 5 wherein the non-nuclease DNA modifying enzyme is fused to an RNA-binding domain.

12. The method of claim 11 wherein the RNA-binding domain derives from a protein selected from the group consisting of the telomerase Sm7, MS2 Coat Protein, PP7 coat protein (PCP), and SfMu phage Com RNA binding protein.

13. The method of claim 11 wherein the RNA-binding domain is the MS2 coat protein variant having an amino acid sequence as set forth in SEQ ID NO:3.

14. The method of claim 5 wherein the plurality of RNA molecules are designed for targeting a plurality of sequences in the DNA nucleic acid molecule encoding for a binding domain of the binding protein.

15. The method of claim 5 wherein the plurality of RNA molecules comprises a programmable guide RNA motif, a CRISPR RNA motif, and a recruiting RNA motif.

16. The method of claim 15 wherein the recruiting RNA motif comprises the telomerase Ku binding motif, the telomerase Sm7 binding motif, the MS2 phage operator stem-loop, the PP7 phage operator stem-loop, or the SfMu phage Com stem-loop.

17. The method of claim 15 wherein the RNA recruiting motif comprises the MS2 Phage Operator Stem Loop as set forth in SEQ ID NO:5.

18. The method of claim 1 wherein the affinity/specificity-based cell selection and immunomagnetic enrichment comprises a step of contacting a post-mutagenesis population of cells with a plurality of multimers made by mixing specific and unspecific target molecules for the binding protein.

19. The method of claim 18 wherein the plurality of multimers are tetramers.

20. The method of claim 19 wherein the tetramers comprise an epitope peptide that is specifically recognized by an antibody that is loaded in a soluble peptide MHC monomer tetramerized with three other soluble peptide MHC monomers that include at least one non-specific MHC peptide monomer.

21. The method of claim 18 wherein the plurality of multimers are conjugated with a label.

22. The method of claim 21 wherein the label is a fluorescent molecule.

23. The method of claim 18 wherein the immunomagnetic enrichment is carried out using magnetic particles to allow cell enrichment by contacting multimers that bind to cells with said magnetic particles.

24. The method of claim 21 wherein the surfaces of the magnetic particles are functionalized to attach binding molecules that bind selectively the label.

25. The method of claim 18 wherein a first round of contacting is carried out with a plurality of multimers having a determined number of specific target molecules and a second round of contacting is carried out with a plurality of multimers having a decreased number of specific target molecules.

26. The method of claim 25 wherein the concentration of the plurality of multimers is progressively decreased between the first round and the second round to increase selection stringency.

27. The method of claim 1, wherein the population of cells express the binding protein at the cell surface.
